(19) United States
(12) Patent Application Publication
(10) Pub. No.: US 2008/0064039 A1
(43) Pub. Date: Mar. 13, 2008

(54) DOMINANT NEGATIVE LIGAND DRUG DISCOVERY SYSTEM

(76) Inventors: Philip T. Pienkos, The Woodlands, TX (US); Daniel J. Monticello, The Woodlands, TX (US)

Correspondence Address: ELMORE PATENT LAW GROUP, PC, 209 MAIN STREET, N. CHELMSFORD, MA, 01863, US

(21) Appl. No.: 11/773,157

(22) Filed: Jul. 3, 2007

Related U.S. Application Data
(60) Provisional application No. 60/818,736, filed on Jul. 6, 2006.

(51) Int. Cl. G01N 33/566; C12Q 1/68; G01N 33/574

(52) U.S. Cl. 435/6; 435/29; 435/7.23; 435/7.92; 436/501

(57) ABSTRACT

The present invention relates to novel methods of designing and optimizing polypeptide based ligands which are useful for altering and/or modulating cellular signaling cascades which have become dysregulated. The therapeutic dominant negative ligands (DNLs) and DNL variants designed by the methods herein have useful applications in medicine, diagnostics and drug discovery.

### RELATED APPLICATION

This application claims the benefit of U.S. Provisional Application No. 60/818,736, filed on Jul. 6, 2006. The entire teachings of the above application are incorporated herein by reference.

### GOVERNMENT SUPPORT

The invention was supported, in whole or in part, by a grant number 2R44CA095930-04 from the National Institutes of Health. The Government has certain rights in the invention.

### FIELD OF THE INVENTION

This invention relates to the field of drug discovery. Specifically it provides biochemical tools and assays that enable the user to identify therapeutic dominant negative ligands and variants thereof that are effective in modulating receptor-mediated pathways, especially those pathways implicated in hyperproliferative conditions such as cancer.

### ABBREVIATIONS

DNL, dominant negative ligand; DBO, domain binding optimization; HER, human epidermal receptor; IR, insulin receptor; IGF insulin-like growth factor; IFN, interferon; hGH, human growth hormone; VEGF, vascular endothelial growth factor; NGF, nerve growth factor; TNF, tumor necrosis factor; GPCR, G-protein coupled receptor.

### BACKGROUND OF THE INVENTION

Interactions between polypeptide ligands and their cognate receptors are critical for a variety of biological processes including maintenance of cellular and organism homeostasis, development, and tumorigenesis. The cell signaling network created by these ligands and receptor interactions is responsible for relaying a majority of the extracellular, intercellular and intracellular signals-handing off signals from one member of the pathway to the next. Modulation of one member of the pathway can be relayed through the signal transduction pathway, resulting in modulation of activities of other pathway members and modulating outcomes of such signal transduction such as affecting phenotypes and responses of a cell or organism to a signal. Diseases and disorders can, and often do, involve dysregulated signal transduction pathways. A goal of therapeutics is to target such dysregulated pathways to restore more normal regulation in the signal transduction pathway. Many ligands can activate multiple independent pathways and the strength of the activation of different pathways can be modulated by the presence or absence of signals generated by other ligands or receptors. Current methods of synthesis and expression of polypeptides involved in cell signaling provide a backdrop for the discovery, investigation and validation of new methods of designing optimized ligands or receptors having therapeutic properties. These optimized molecules can then be exploited in the areas of drug discovery and medicine, including gene therapy.

### SUMMARY OF THE INVENTION

Accordingly, it is an object herein to provide novel ligands and ligand variants for use, among other things, as therapeutics and methods for designing and identifying said ligand therapeutics.

One aspect of the invention relates to a method for designing therapeutic dominant negative ligands (DNLs) and variants thereof comprising selecting a druggable ligand wherein the known or predicted structure of the druggable ligand presents or contains two or more receptor binding surfaces. Once a druggable ligand is selected and optionally optimized, domain binding optimization (DBO) is performed on the druggable ligand by a method which comprises making one or more modifications to one or more features at a first receptor binding surface of the druggable ligand to disrupt binding of the druggable ligand to a first target receptor domain, and making one or more modifications to one or more features at a second receptor binding surface of the druggable ligand to enhance binding of the druggable ligand to a second target receptor domain. Druggable ligands of the invention are selected from either known receptor ligands or a polypeptide sequence designed to function as a druggable ligand.

The druggable ligands, once having undergone domain binding optimization, are then assayed for their ability to inhibit a biological activity in one or more cell lines wherein the biological activity is selected from the group consisting of a receptor-mediated pathology, receptor-mediated cell signaling, cell growth, cell proliferation and tumor growth. In one embodiment of the invention, the inhibited biological activity is receptor-mediated cell signaling. This inhibition of receptor-mediated cell signaling may result in ablation of downstream signaling by a receptor and this effect can be determined by measuring altered phosphorylation states of one or more proteins.

In one embodiment of the invention the inhibition of receptor-mediated cell signaling is measured using autophosphorylation assays or gene expression assays.

In one embodiment of the invention the inhibition of biological activity is panoramic over two or more receptors. Further, the level or degree of panoramic inhibition of biological activity may be or is substantially the same against said two or more receptors.

In one embodiment of the invention the inhibited biological activity is a receptor-mediated pathology. Receptor-mediated pathologies may be selected from the group consisting of cancer, inflammation, cardiovascular disease, hyperlipidemia, glucose dysregulation, epilepsy, allergies, chronic pain, Alzheimers disease, metabolic syndrome, cortisol resistance, Crohn disease and Huntington disease.

In one embodiment of the invention the one or more cell lines comprises a cancer cell line. Cancer cell lines include, but are not limited to cancer of lung, breast, liver, heart, bone, blood, colon, brain, skin, kidney, pancreatic, ovarian, uterine and prostate. MCF-7 represents a breast cancer cell line.

In one aspect a method further comprising the step of identifying druggable ligands capable of inhibiting a biological activity as therapeutic dominant negative ligands.

In one embodiment of the invention are provided methods comprising making modifications to one or more features of the druggable ligands to alter one or more properties of the druggable ligands, said properties selected from the group consisting of optimal pH or pH-activity, digestibility, antigenicity, the amphipathic properties, ligand-receptor interactions, thermal or kinetic stability, solubility, folding, posttranslational modification, hydrophobicity, hydrophilicity, isoelectric point, protease resistance, and aromaticity.

In one embodiment of the invention are methods wherein the disruption or enhancement of binding of the druggable ligand to a said first or a said second target receptor domain is determined by measuring the binding affinity of the druggable ligand to one or more molecules selected from the group consisting of native target receptors containing the target receptor domain, isolated target receptor domains and representative target receptor moieties.

In one embodiment of the invention said first and said second target receptor domains are located in the same receptor.

In one embodiment of the invention the target receptor is selected from the group consisting of HER receptors, insulin receptors, IGF receptors, interferon receptors, hGH receptors, VEGF receptors, NGF receptors, TNF receptors, G-protein coupled receptors and any other receptor pathway known to operate, be triggered or function via polypeptide ligand binding.

In one embodiment of the invention the target receptor is membrane bound but it is understood that the receptor may be localized to any cell, or cellular organelle including the nuclear membrane. Furthermore the receptor may be soluble in nature having little or no membrane anchoring.

In one embodiment of the invention, the modifications made result in the production of a library of modified polypeptides. In one embodiment of the invention, the modifications made result from a starting library of modified polypeptides. The library of modified polypeptides may comprise a phage library or any other selection or grouping of polypeptide sequences independent of the manner in which they were generated.

In one embodiment, binding of ligands and receptors is determined using phage ELISA. It is understood that many binding assays are known in the art and these are also contemplated by the present invention. The methods of the present invention further contemplate the step of repeating the phage panning of the druggable ligands. This repetition may be performed to optimize any or all of the properties of the druggable ligand or DNL being investigated. It may also be performed in order to increase the population of domain binding optimized druggable ligands.

In one embodiment of the invention, the one or more modifications are selected from the group consisting of randomization of one or more features, duplication of one or more features, alteration of length, alteration of electronic charge or aromaticity, and any combination thereof. It is contemplated by the present invention that, among other modifications, alteration of length can be a truncation, internal deletion or insertion. Other alterations in length may simply result from the synthesis of less than a full-length ligand.

In one embodiment of the invention, the one or more features are selected from the group consisting of surface manifestations, local conformational shape, fold, loops, half-loops, domains, half-domains, sites and termini.

In one embodiment of the invention the methods may further comprise the step of rational redesign wherein the steps of selecting druggable ligands and the modifications made during DBO to said selected druggable ligands are performed iteratively, either alone or in combination.

The present invention encompasses any of the therapeutic DNLs or DNL variants produced by the methods disclosed herein.

In one embodiment of the invention are methods of identifying anticancer agents comprising assaying therapeutic DNLs or DNL variants designed by the methods described herein in a tumor xenograft system wherein a measured reduction in tumor growth rate, tumor size or tumor metastasis represents a positive hit as a candidate cancer therapeutic.

### DETAILED DESCRIPTION OF THE INVENTION

A description of embodiments of the invention follows.

The present invention results from the discovery of a novel method of designing and optimizing polypeptide based ligands which are then useful for altering and/or modulating cellular signaling cascades which have become dysregulated. As such, the present invention encompasses therapeutic dominant negative ligands (DNLs) and variants thereof and methods for their design and use in medicine, diagnostics and drug discovery. The DNLs of the present invention possess optimized properties as well as dominant negative activity toward receptors.

Designing Dominant Negative Ligands (DNLs) and Variants

One aspect of the invention includes a method for designing therapeutic dominant negative ligands (DNLs) and variants thereof. This method comprises selecting a druggable ligand and performing domain binding optimization (DBO) on the selected druggable ligand. Optionally, druggable ligands may undergo optimization prior to DBO. Once a druggable ligand has undergone DBO, the ligand can then be assayed for biological activity as a dominant negative ligand. Optionally, it may be desired to assay the druggable ligand for biological activity as a dominant negative ligand prior to, between or during DBO. Those druggable ligands capable of inhibiting a biological activity as dominant negative ligands are identified or termed therapeutic dominant negative ligands. The therapeutic DNLs identified by the methods of the present invention are useful in the treatment of diseases or disorders resulting from or characterized by dysregulated receptor-mediated cell signaling events.

Selection of a Druggable Ligand

As a starting point, the design method disclosed herein begins with the selection of a druggable ligand. “Druggable ligands” include any ligand which may serve as a starting ligand for the methods of the present invention. These ligands are selected from known receptor ligands or any polypeptide sequence designed to function as a druggable ligand. For example, in copending application U.S. application Ser. No. 11/172,611, filed Jun. 30, 2005, the entire teachings of which are incorporated herein by reference, known HER ligands are used as starting points for investigation. The known or predicted structure of the selected druggable ligands of the present invention must present, contain or be designed to contain two or more receptor binding surfaces. These receptor binding surfaces may be the same in either structure or functional characteristics, but it is not necessary that they be the same. As used herein the term “same” means identical in relation to the property being considered. As used herein the term “similar” means alike in at least one way. Sameness or similarity may be used in the context of structure or function.

Any polypeptide based molecule meeting the criterion defined above is considered a druggable ligand. Receptor binding surfaces may be distinct and separable surfaces, adjacent surfaces or may overlap in space or sequence (i.e., may each utilize the same or common amino acids as a component of the surface).

As the term is used herein, “receptor binding surfaces” are motifs found in druggable ligands and DNLs of the invention which serve as the site of interaction between a ligand and a receptor. The receptor binding surfaces may be defined by a particular amino acid sequence or result from protein folding, e.g., when surfaces are created by nonadjacent amino acids coming into proximity due to electrostatic or thermodynamic energy minimization of the overall sequence of the polypeptide to produce secondary and/or tertiary protein structures.

The corresponding motif in a receptor which serves as the site of interaction between a druggable ligand or DNL ligand and receptor is herein referred to as the “target receptor domain.”

As used herein the term “ligand” is used to designate a polypeptide based molecule capable of specific binding to a receptor as herein defined. The definition includes any native ligand for a receptor or any region or derivative thereof retaining at least a qualitative receptor binding ability. Specifically excluded from this definition are antibodies to a receptor and noncovalent conjugates of an antibody and an antigen for that antibody.

The terms “native ligand” and “wild-type ligand” are used interchangeably and refer to an amino acid sequence of a ligand occurring in nature (“native sequence ligand”), including mature, pre-pro and pro forms of such ligands, purified from natural source, chemically synthesized or recombinantly produced. Native ligands that can activate receptors are well known in the art or can be prepared by art known methods.

Regarding the dominant negative ligands of the present invention, the term “dominant negative” is used to describe that type of ligand, when altered or modified to differ from the native or wild-type ligand in any respect, results in a ligand that retains binding affinity for a wild-type binding partner (e.g., a receptor) but inhibits the function or signaling of the wild-type binding partner.

The present invention contemplates the design of DNLs, as that term applies to the aforementioned functional properties, as well as “DNL variants” which have as their design reference point, DNLs. These DNL variants may be the result of further optimization of properties in addition to or beyond binding and signal inhibition. For example, once optimized over a first DNL, a DNL variant may then be the starting point for further optimization meaning that, in the design scheme, the DNL variant would then become the starting DNL. Therefore, a “DNL” can, in certain contexts, be construed as a “DNL variant” and vice versa. Furthermore, when used as a starting or reference point for design, a DNL or DNL variant may also be referred to or considered a druggable ligand.

As used herein the term “dominant negative ligand activity” refers to the functions associated with dominant negative ligands (e.g., binding a receptor at its normal ligand binding site but inhibiting a function of the receptor).

The druggable ligands and DNLs of the present invention are polypeptide based molecules. These molecules may be “peptides,” “polypeptides,” or “proteins.” While it is known in the art that these terms imply relative size, these terms as used herein should not be considered limiting with respect to the size of the various polypeptide based molecules referred to herein and which are encompassed within this invention. Thus, any amino acid sequence comprising at least one of the DNLs or their receptor binding surfaces disclosed herein, and which binds to any receptor is within the scope of this invention.

The terms “amino acid” and “amino acids” refer to all naturally occurring L-alpha-amino acids. The amino acids are identified by either the one-letter or three-letter designations as listed in Table 1.

TABLE 1
Naturallly occurring amino acids

| Three letter | One letter | Amino acid |
|---|---|---|
| Asp | D | aspartic acid |
| Ile | I | isoleucine |
| Thr | T | threonine |
| Leu | L | leucine |
| Ser | S | serine |
| Tyr | Y | tyrosine |
| Glu | E | glutamic acid |
| Phe | F | phenylalanine |
| Pro | P | proline |
| His | H | histidine |
| Gly | G | glycine |
| Lys | K | lysine |
| Ala | A | alanine |
| Arg | R | arginine |
| Cys | C | cysteine |
| Trp | W | tryptophan |
| Val | V | valine |
| Gln | Q | glutamine |
| Met | M | methionine |
| Asn | N | asparagine |

The amino acid sequences of the DNLs of the invention may comprise naturally occurring amino acids and as such may be considered to be proteins, peptides, polypeptides, or fragments thereof. Alternatively, the DNLs may comprise both naturally and non-naturally occurring amino acids.

The term “amino acid sequence variant” refers to molecules with some differences in their amino acid sequences as compared to a native sequence. The amino acid sequence variants may possess substitutions, deletions, and/or insertions at certain positions within the amino acid sequence of a native ligand. Ordinarily, variants will possess at least about 70% homology to a native ligand, and preferably, they will be at least about 80%, more preferably at least about 90% homologous to a native ligand.

“Homology” as it applies to amino acid sequences is defined as the percentage of residues in the candidate amino acid sequence that are identical with the residues in the amino acid sequence of a native ligand after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” is meant the corresponding ligand or receptor of other species having substantial identity to human wild-type ligand or receptors.

“Analogs” is meant to include polypeptide variants which differ by one or more amino acid alterations, e.g., substitutions, additions or deletions of amino acid residues that still maintain the dominant negative properties of the parent polypeptide. As stated above, parent molecules (i.e., the reference point for comparison) may comprise druggable ligands, DNLs or DNL variants.

As described herein, the DNLs and DNL variants produced by the methods of the present invention, their homologs and analogs may have substantial identity to wild-type ligands. As used herein, “substantial identity” means at least 60% sequence identity, preferably at least 70% identity, preferably at least 80% and more preferably at least 90% sequence identity to the amino acid sequence of wild-type human ligand (or domains thereof in the instance where the variant is a chimera produced by swapping domains), while maintaining dominant negative activity. In other embodiments, the DNLs and their variants of the present invention have at least 91%, at least 92%, at least 93%, at least 94%, at least 95% at least 96%, at least 97%, or at least 98% amino acid identity to the amino acid sequence of wild-type human ligand, while maintaining dominant negative ligand activity.

The percent identity of two amino acid sequences can be determined by aligning the sequences for optimal comparison purposes (e.g., gaps can be introduced in the sequence of a first sequence). The amino acids at corresponding positions are then compared, and the percent identity between the two sequences is a function of the number of identical positions shared by the sequences (i.e., % identity=# of identical positions/total # of positions×100). The actual comparison of the two sequences can be accomplished by well-known methods, for example, using a mathematical algorithm. A preferred, non-limiting example of such a mathematical algorithm is described in Karlin et al., Proc. Natl. Acad. Sci. USA, 90:5873-5877 (1993). Such an algorithm is incorporated into the BLASTN and BLASTX programs (version 2.2) as described in Schaffer et al, Nucleic Acids Res. 29:2994-3005 (2001).

The term “derivative” is used synonymously with the term “variant” and refers to a molecule that has been modified or changed in any way relative to a reference molecule or starting molecule. As used herein derivative and variant dominant negative ligands are polypeptide based molecules which are modified, altered, improved or optimized relative to a starting parent molecule.

The present invention contemplates several types of dominant negative ligand variants and derivatives. These include substitutional, insertional, deletion and covalent variants and derivatives.

As such, included within the scope of this invention are polypeptide based molecules containing substitutions, insertions and/or additions, deletions and covalently modifications. For example, sequence tags or amino acids, such as one or more lysines, can be added to the peptide sequences of the invention (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” are those with one or more amino acids in the native or starting amino acid sequence removed. Ordinarily, deletional variants will have one or more amino acids deleted in a particular region of the molecule.

“Covalent derivatives” include modifications of a native or starting ligand with an organic proteinaceous or non-proteinaceous derivatizing agent, and post-translational modifications. Covalent modifications are traditionally introduced by reacting targeted amino acid residues of the ligand with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-ligand antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the ligands used in accordance with the present invention.

Other post-translational modifications include hydroxylation of proline and lysine, phosphorylation of hydroxyl groups of seryl or threonyl residues, methylation of the .alpha.-amino groups of lysine, arginine, and histidine side chains (T. E. Creighton, Proteins: Structure and Molecular Properties, W.H. Freeman & Co., San Francisco, pp. 79-86 (1983)).

Covalent derivatives specifically include fusion molecules in which ligands of the invention are covalently bonded to a nonproteinaceous polymer. The nonproteinaceous polymer ordinarily is a hydrophilic synthetic polymer, i.e. a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyvinylalkylene ethers such a polyethylene glycol, polypropylene glycol. The ligands may be linked to various nonproteinaceous polymers, such as polyethylene glycol, polypropylene glycol or polyoxyalkylenes, in the manner set forth in U.S. Pat. No. 4,640,835; 4,496,689; 4,301,144; 4,670,417; 4,791,192 or 4,179,337.

In one embodiment of the invention, binding is ablated using a polyethylene glycol (PEG) modification. For example, a lysine located in a receptor binding surface which is important for binding may be PEGylated to attenuate or ablate the binding properties of that surface. It is contemplated to be within the scope of the invention that any amino acid may be modified in such a way as to ablate binding. In addition, it is also within the scope of the invention to use PEGylation to improve properties such as half-life and to reduce immunogenicity.

Post-translational variants also include glycosylation variants. The term “glycosylation variant” is used to refer to a ligand having a glycosylation profile different from that of a native or starting ligand. Any difference in the location and/or nature of the carbohydrate moieties present in a dominant negative ligand as compared to its native or starting counterpart is within the scope herein.

The glycosylation pattern of native or starting ligands can be determined by well known techniques of analytical chemistry, including HPAE chromatography (Hardy, M. R. et al., Anal. Biochem. 170, 54-62 (1988)), methylation analysis to determine glycosyl-linkage composition (Lindberg, B., Meth. Enzymol. 28. 178-195 (1972); Waeghe, T. J. et al., Carbohydr. Res. 123, 281-304 (1983)), NMR spectroscopy, mass spectrometry, etc. For ease, changes in the glycosylation pattern of a native or starting ligand are usually made at the DNA level, essentially using the techniques known in the art with respect to the amino acid sequence variants.

Carbohydrate moieties present on a ligand may also be removed chemically or enzymatically. Chemical or enzymatic coupling of glycosides to the ligands of the present invention may also be used to modify or increase the number or profile of carbohydrate substituents. These methods are described in WO 87/05330 (published 11 Sep. 1987), and in Aplin and Wriston, CRC Crit. Rev, Biochem., pp. 259-306.

Glycosylation variants of the ligands herein can also be produced by exploiting in vivo methods such as the normal processes of an appropriate host cell. Yeast, for example, introduce glycosylation which varies significantly from that of mammalian systems. Similarly, mammalian cells having a different species (e.g. hamster, murine, insect, porcine, bovine or ovine) or tissue (e.g. lung, liver, lymphoid, mesenchymal or epidermal) origin than the source of the ligand, are routinely screened for the ability to introduce variant glycosylation.

Amino acid sequences of the druggable ligands, DNLs and DNL variants of the invention may be obtained through various means such as chemical synthesis, phage display, cleavage of proteins or polypeptides into fragments, or by any means which amino acid sequences of sufficient length to possess selected properties may be made or obtained.

In one embodiment, the DNL variants of the invention are produced by expression in a suitable host of a gene coding for the relevant DNL variant. Such a gene is most readily prepared by site-directed mutagenesis of the wild-type gene, a technique well known in the art.

As such, the present invention also provides nucleic acid molecules encoding a DNL or DNL variant of the invention. The nucleic acid molecules of the present invention can be RNA, for example, mRNA, or DNA. DNA molecules can be double-stranded or single-stranded. The nucleic acid molecule can also be fused to a marker sequence, for example, a sequence that encodes a polypeptide to assist in isolation or purification of the polypeptide. Such sequences include, but are not limited to, those that encode a glutathione-S-transferase (GST) fusion protein, those that encode a hemagglutinin A (HA) polypeptide marker from influenza, and sequences encoding a His tag.

It will be appreciated by those skilled in the art that the design of the expression vector can depend on such factors as the choice of the host cell to be transformed and the level of expression of DNL desired. The expression vectors of the invention can be introduced into host cells to thereby produce the modified polypeptides of the invention, including fusion polypeptides, encoded by nucleic acid molecules as described herein. Molecular biology techniques for carrying out recombinant production of the modified polypeptides of the invention are well known in the art and are described for example, in, Sambrook, et al., Molecular Cloning: A Laboratory Manual (Cold Spring Harbor Lab Press; 3^{rd} ed., 2000).

Alternatively, the DNL variants of the invention may be produced in whole or in part by chemical synthetic techniques such as by a Merrifield-type synthesis (J. Am. Chem. Soc. 85:2149 (1963), although other equivalent chemical syntheses known in the art may be used. Solid-phase synthesis is initiated from the C-terminus of the peptide by coupling a protected alpha-amino acid to a suitable resin. The amino acids are coupled the peptide chain using techniques well known in the art for the formation of peptide bonds. Chemical synthesis of all or a portion of a DNL of the invention may be particularly desirable in the case of the use of a non-naturally occurring amino acid substituent in the DNL variant.

Modifications and Manipulations

In order to design effective therapeutic dominant negative ligands according to the methods of the invention, it is necessary to optimize the druggable ligands selected. This optimization may include modifications to the selected druggable ligands prior to domain binding optimization or afterwards. The process of optimizing may be iterative, requiring several rounds of modifications to optimize each of a number of properties of the DNL or it may occur step-wise in a sequential manner. Modifications may be made singly, or combinatorially to improve or alter one or more properties of the molecules.

In one embodiment of the invention are provided methods comprising making modifications to one or more features of the druggable ligands to alter one or more properties of the druggable ligands, said properties selected from the group consisting of optimal pH or pH-activity, digestibility, antigenicity, half-life, bioavailability, the amphipathic properties, ligand-receptor interactions, thermal or kinetic stability, solubility, folding, posttranslational modification, hydrophobicity, hydrophilicity, and any combination thereof. It will be understood by those of skill in the art that the properties listed represent considerations in developing therapeutics, diagnostics and research tools and that other properties of molecules may also need to be considered and optimized depending on the particular application. As used herein the term “optimized or optimization” refers to the modification or alteration of a molecule such that one or more characteristics of the molecule are improved for a particular purpose as compared to a starting molecule. “Modification” is the result of modifying wherein the thing being modified is changed in form or character. The molecules of the present invention being optimized via modifications include druggable ligands, DNLs and their variants. For the purposes of the instant invention, these molecules are being optimized for the purpose of creating therapeutic, diagnostic or research reagents.

The modifications of the present invention are herein made to one or more features of the druggable ligands, DNLs or DNL variants. “Features” are defined as distinct amino acid sequence-based components of a molecule. Features of the druggable ligands, DNLs and DNL variants of the present invention include surface manifestations, local conformational shape, folds, loops, half-loops, domains, half-domains, sites, termini or any combination thereof.

As used herein the term “surface manifestation” refers to a polypeptide based component of a druggable ligand or DNL appearing on an outermost surface.

As used herein the term “local conformational shape” means a polypeptide based structural manifestation of a druggable ligand or DNL which is located within a definable space of the druggable ligand or DNL.

As used herein the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol 266 (4): 814-830; 1997).

As used herein the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein the term “domain” refers to a motif of a polypeptide having one or more identifiable structural or functional characteristics or properties (e.g., binding capacity, serving as a site for protein-protein interactions.

As used herein the term “half-domain” means portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein the terms “site” is used synonymous with “amino acid residue” and “amino acid side chain”. A site represents a position within a peptide or polypeptide that may be modified, manipulated, altered, derivatized or varied within the polypeptide based molecules of the present invention.

As used herein the terms “termini or terminus” refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH2)) and a C-terminus (terminated by an amino acid with a free carboxyl group (COOH)). Druggable ligands are in some cases made up of multiple polypeptide chains brought together by disulfide bonds or by non-covalent forces (multimers, oligomers). These sorts of ligands will have multiple N- and C-termini. Alternatively, the termini of the polypeptides may be modified such that they begin or end, as the case may be, with a non-polypeptide based moiety such as an organic conjugate.

Once any of the features have been identified or defined as a component of a molecule of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

Domain Binding Optimization (DBO)

Once a druggable ligand has been selected, and optionally modified or optimized, domain binding optimization (DBO) of the druggable ligand is performed.

As used herein “domain binding optimization” involves making one or more modifications or manipulations as described above to one or more features at a first receptor binding surface of the druggable ligand to disrupt binding of the druggable ligand to a first target receptor domain, and making one or more modifications to one or more features at a second receptor binding surface of the druggable ligand to enhance binding of the druggable ligand to a second target receptor domain.

As stated above, a “target receptor domain” is the corresponding motif in a receptor which serves as the site of interaction between a druggable ligand or DNL ligand and receptor.

As used herein the terms “receptor” and “target receptor” may be used interchangeably and refer to the member of the ligand-receptor binding pair which effects alteration of downstream signaling events.

For the purpose of the present invention the receptor can be any receptor selected from membrane-bound (including cell surface, nuclear and organelle surface) or soluble receptors having a receptor activity irrespective of the actual mechanism by which the receptor-effected activity is induced. In one embodiment of the invention the target receptor is membrane bound but it is understood that such membrane-bound receptor may be localized to any cell, or cellular organelle including the nuclear membrane. Furthermore, the receptor may be soluble in nature having little or no membrane anchoring.

The definition of receptor includes cell-surface receptors that are normally activated a) by monovalent ligands (ligands with one receptor binding surface), b) by polyvalent ligands (ligands with two or more receptor binding surfaces), or c) by interaction of the ligand with a receptor dimer and subsequent intracomplex conformational change.

Receptor binding surfaces in ligands and target receptor domains in receptors can be determined by methods known in the art, including computational analysis (e.g., molecular modeling), X-ray studies, mutational analyses, antibody binding studies, and random peptide library panning and binding studies. The mutational approaches include the techniques of site-directed mutagenesis, random saturation mutagenesis coupled with selection of escape mutants, insertional mutagenesis, and homolog-scanning mutagenesis (replacement of sequences from human ligands, which bind the corresponding receptor, with unconserved sequences of a corresponding ligand from another animal species, e.g. mouse, which do not bind the human receptor).

In one embodiment of the invention said first and said second target receptor domains are located in the same receptor. However the target receptor domains may be located in separate molecules of the same receptor type or in two separate types of receptor molecules. Furthermore, for the purposes of the binding assays, the entire receptor need not be used and binding need only be evaluated using a molecule comprising the target receptor domain. As such, in one embodiment of the invention are methods wherein the disruption or enhancement of binding of the druggable ligand to a said first or a said second target receptor domain is determined by measuring the binding affinity of the druggable ligand to one or more molecules selected from the group consisting of native target receptors containing the target receptor domain, isolated target receptor domains and representative target receptor moieties.

In one embodiment of the invention the target receptor is selected from the group consisting of HER receptors, insulin receptors, IGF receptors, interferon receptors, hGH receptors, VEGF receptors, NGF receptors, TNF receptors, G-protein coupled receptors (GPCRs) and any other receptor pathway known to operate, be triggered by, or function via ligand binding.

Binding Studies

As domain binding optimization involves modification of the binding properties of the druggable ligands, it is necessary to perform certain binding assays to assess the resultant binding properties of the ligand after DBO. It is understood that many binding assays for assessing protein-protein binding and ligand-receptor binding are known in the art and within the ability of one of ordinary skill in the art.

The DNLs provided by this invention should have an affinity for a receptor sufficient to provide adequate binding for the intended purpose. Thus, for use as a therapeutic, the peptide, polypeptide, or protein provided by this invention should have an affinity (Kd) of between about 1-1000 nM for the target receptor. More preferably the affinity is 10 nM. Most preferably, the affinity is 1 nM. For use as a reagent in a competitive binding assay to identify other ligands, the amino acid sequence preferably has affinity for the receptor higher than or equal to the authentic ligand.

As used herein the term “binding” includes the formation of one or more ionic, covalent, hydrophobic, electrostatic, or hydrogen bonds between a receptor binding surface of the druggable ligands or DNLs of the invention and one or more amino acids of a target receptor domain of a target receptor. Binding can be considered “tight” if the DNL is not substantially displaced in an in vitro assay. The DNL is not substantially displaced if at least 50%, preferably at least 70%, more preferably at least about 90%, such as 100%, of the DNL remains bound to a receptor or receptor moiety when competitively challenged with a native ligand. Binding can also be considered tight if the DNL substantially displaces the native ligand from the receptor. The DNL substantially displaces the native ligand if at least 50%, preferably at least 70%, more preferably at least about 90%, such as 100%, of the native ligand is displaced from the receptor.

The binding or bioactive activity of a DNL or DNL variant of the invention can further be assessed by any other suitable assay or other method, wherein the results or activity of such assay are compared to the binding or receptor activity from an assay which measures the binding or receptor activity of wild-type human ligands and receptors.

In one embodiment of the invention, binding studies are performed on libraries of compounds of the invention. Methods of library production can also be used to create the druggable ligand starting molecules of the invention.

In one embodiment of the invention, the modifications made to the druggable ligands or DNLs result in or from the production of a library of modified polypeptides. The library of modified polypeptides may comprise a phage library or any other selection or grouping of polypeptide sequences independent of the manner in which they were generated.

As used herein, the term “library” means a collection of molecules. A library can contain a few or a large number of different molecules, varying from about two to about 10¹⁵ molecules or more. The chemical structure of the molecules of a library can be related to each other or be diverse. If desired, the molecules constituting the library can be linked to a common or unique tag, which can facilitate recovery and/or identification of the molecule.

Phage Panning

Methods for preparing libraries containing diverse populations of various types of molecules such as peptides, proteins, peptoids and peptidomimetics are well known in the art and various libraries are commercially available (see, for example, Ecker and Crooke, Biotechnology 13:351-360 (1995), and Blondelle et al., Trends Anal. Chem. 14:83-92 (1995), and the references cited therein, each of which is incorporated herein by reference; see, also, Goodman and Ro, Peptidomimetics for Drug Design, in “Burger's Medicinal Chemistry and Drug Discovery” Vol. 1 (ed. M. E. Wolff; John Wiley & Sons 1995), pages 803-861, and Gordon et al., J. Med. Chem. 37:1385-1401 (1994), each of which is incorporated herein by reference). Where a molecule is a peptide, protein or fragment thereof, the molecule can be produced in vitro directly or can be expressed from a nucleic acid, which can be produced in vitro. Methods of synthetic peptide and nucleic acid chemistry are well known in the art.

In addition, a library of molecules can be a library of nucleic acid molecules, which can be DNA, RNA or analogs thereof. For example, a cDNA library can be constructed from mRNA collected from a cell, tissue, organ or organism of interest, or by collecting genomic DNA, which can be treated to produce appropriately sized fragments using restriction endonucleases or methods that randomly fragment genomic DNA. A library comprising RNA molecules also can be constructed by collecting RNA from cells or by synthesizing the RNA molecules chemically. Methods for producing such libraries are well known in the art (see, for example, Sambrook et al., Molecular Cloning: A laboratory manual (Cold Spring Harbor Laboratory Press 1989), which is incorporated herein by reference). Diverse libraries of nucleic acid molecules can be made using solid phase synthesis, which facilitates the production of randomized regions in the molecules. If desired, the randomization can be biased to produce a library of nucleic acid molecules containing particular percentages of one or more nucleotides at a position in the molecule (U.S. Pat. No. 5,270,163, issued Dec. 14, 1993, which is incorporated herein by reference).

In one embodiment of the invention, binding of ligands and receptors is determined using phage panning of a library of ligands. For example, an assay may be performed screening a druggable ligand library or DNL library which was produced via phage expression.

The screening of very large protein libraries has been accomplished by a variety of techniques that rely on the display of proteins on the surface of viruses or cells. The underlying premise of display technologies is that proteins engineered to be anchored on the external surface of biological particles (i.e., cells or viruses) are directly accessible for binding to ligands without the need for lysing the cells. Viruses or cells displaying proteins with affinity for a ligand can be isolated in a variety of ways including sequential adsorption/desorption form immobilized ligand, by magnetic separations or by flow cytometry (Ladner et al. 1993, U.S. Pat. No. 5,223,409, Ladner et al. 1998, U.S. Pat. No. 5,837,500, Georgiou et al. 1997, Shusta et al. 1999).

The most widely used display technology for protein library screening applications is phage display. Phage display is a well-established and powerful technique for the discovery of proteins that bind to specific ligands and for the engineering of binding affinity and specificity (Rodi and Malowski, Curr. Opin. Biotechnol., 10:87-93; 1999; Wilson and Finlay, Canadian Journal of Microbiology, 44:313-329; 1998). In phage display, a gene of interest is fused in-frame to phage genes encoding surface-exposed proteins, most commonly pIII. The gene fusions are translated into chimeric proteins in which the two domains fold independently. Phage displaying a protein with binding affinity for a ligand can be readily enriched by selective adsorption onto immobilized ligand, a process known as “panning”. The bound phage is desorbed from the surface, usually by acid elution, and amplified through infection of E. coli cells. Usually, 3-6 rounds of panning and amplification are sufficient to select for phage displaying specific polypeptides, even from very large libraries with diversities up to 10¹⁵. Each round of panning enriches the pool of clones in favor of the tightest-binding ligands. Because each phage particle contains both the displayed peptide and the DNA encoding it, the selected peptides can be readily identified by DNA sequencing. Several variations of phage display for the rapid enrichment of clones displaying tightly binding polypeptides have been developed (Duenas and Borrebaeck, 1994; Malmborg et al., 1996; Kjaer et al., 1998; Burioni et al., 1998; Levitan, 1998; Mutuberria et al., 1999; Johns et al., 2000).

The phage panning methods of the present invention involve introduction of an oligonucleotide encoding the DNL and DNL variants of the present invention for expression on the phage particle surface and panning the phage particles against the target receptors or receptor moieties. Phage panning may be used in conjunction with other binding assays such as enzyme linked immunosorbent assay (ELISA) methods.

The methods of the present invention further contemplate the step of repeating the phage panning of the druggable ligands. This repetition may be performed to optimize any or all of the properties of the druggable ligand or DNL being investigated. It may also be performed in order to increase the population of domain binding optimized druggable ligands.

Rational Redesign

In one embodiment of the invention the methods may further comprise the step of rational redesign wherein the steps of selecting druggable ligands and the modifications made in the DBO step to the selected druggable ligands are performed iteratively, either alone or in combination.

Dominant Negative Activity of DNLs

The druggable ligands and DNLs of the present invention can be assayed for inhibition of receptor-mediated bioactivity in one or more cell lines using a number of known methods, assays, devices and kits well known in the art.

In one embodiment of the invention the one or more cell lines comprises a cancer cell line. Cancer cell lines include, but are not limited to lung, breast, liver, heart, bone, blood, colon, brain, skin, kidney, pancreatic, ovarian, uterine and prostate or any cells isolated from tissues or tumors of the cancers listed herein.

In one embodiment of the invention are methods of identifying anticancer agents comprising assaying therapeutic DNLs or DNL variants designed by the methods described herein in a tumor xenograft system wherein a measured reduction in tumor growth rate, tumor size or tumor metastasis represents a positive hit as a candidate cancer therapeutic.

In one embodiment the disease associated with dysregulated cell signaling is a tumor. In particular the tumor is a solid tumor and/or blood or lymphatic node cancer. More specifically, tumors which can be of epithelial or mesodermal origin, can be benign or malignant types of tumors in organs such as lungs, prostate, urinary bladder, kidneys, esophagus, stomach, pancreas, brain, ovaries, skeletal system, with adenocarcinoma of breast, prostate, lungs and intestine, bone marrow cancer, melanoma, hepatoma, ear-nose-throat tumors in particular being explicitly preferred as members of so-called malignant tumors.

According to the invention, the group of blood or lymphatic node cancer types includes all forms of leukemias (e.g. in connection with B cell leukemia, mixed-cell leukemia, null cell leukemia, T cell leukemia, chronic T cell leukemia, HTLV-II-associated leukemia, acute lymphocytic leukemia, chronic lymphocytic leukemia, mast cell leukemia, and myeloid leukemia) and lymphomas.

Examples of mesenchymal malignant tumors (so-called bone and soft-tissue sarcomas) are: fibrosarcoma; malignant histiocytoma; liposarcoma; hemangiosarcoma; chondrosarcoma and osteosarcoma; Ewing sarcoma; leio- and rhabdomyosarcoma, synovialsarcoma; carcinosarcoma.

Also contemplated within the scope of the invention are neoplasms. Neoplasms include: bone neoplasms, breast neoplasms, neoplasms of the digestive system, colorectal neoplasms, liver neoplasms, pancreas neoplasms, hypophysis neoplasms, testicle neoplasms, orbital neoplasms, neoplasms of head and throat, of the central nervous system, neoplasms of the hearing organ, pelvis, respiratory tract and urogenital tract.

In another embodiment the cancerous disease or tumor being treated or prevented is selected from the group of: tumors of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs, comprising non-parvicellular bronchial carcinomas, parvicellular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarian carcinoma, tumors of the uterine tube, tumors of the abdominal cavity, mammary carcinomas, tumors of the endocrine organs, comprising tumors of the thyroid, parathyroid, adrenal cortex, endocrine pancreas tumors, carcinoid tumors and carcinoid syndrome, multiple endocrine neoplasias, bone and soft-tissue sarcomas, mesotheliomas, skin tumors, melanomas comprising cutaneous and intraocular melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, Hodgkin's disease, leukemias comprising acute leukemias, chronic myeloid and lymphatic leukemias, plasma cell neoplasms, myelodysplasia syndromes, paraneoplastic syndromes, metastases with unknown primary tumor (CUP syndrome), peritoneal carcinomatosis, immunosuppression-related malignancy comprising AIDS-related malignancies such as Kaposi sarcoma, AIDS-associated lymphomas, AIDS-associated lymphomas of the central nervous system, AIDS-associated Hodgkin disease, and AIDS-associated anogenital tumors, transplantation-related malignancy, metastasized tumors comprising brain metastases, lung metastases, liver metastases, bone metastases, pleural and pericardial metastases, and malignant ascites.

According to the present invention, the biological activity being assayed includes, but is not limited to, a receptor-mediated pathology such as any of the diseases or conditions noted herein, receptor-mediated cell signaling, cell growth, cell proliferation and tumor growth.

As used herein the term “receptor-mediated” refers to any phenomenon or condition, the occurrence of which can be linked or traced to the function or activity of a receptor, as that term is defined herein.

In one embodiment of the invention the inhibited biological activity is a receptor-mediated pathology selected from the group consisting of cancer (including all those identified hereinabove), inflammation, cardiovascular disease, hyperlipidemia, glucose dysregulation, epilepsy, allergies, chronic pain, Alzheimers disease, metabolic syndrome, cortisol resistance, Crohn disease and Huntington disease.

In one embodiment of the invention, the inhibited biological activity is receptor-mediated cell signaling. This inhibition of receptor-mediated cell signaling may result in ablation of downstream signaling by a receptor and this effect can be determined by measuring altered phosphorylation states of one or more proteins.

According to the present invention, inhibition of receptor-mediated cell signaling can be measured using autophosphorylation assays or gene expression assays. Methods of measuring and quantifying cell signaling cascades are known in the art as are methods to measure gene expression either by measuring mRNA (e.g., RT-PCR) or measuring protein levels (e.g., Western blot analysis).

It is within the scope of the present invention to design therapeutic DNLs that are capable of dominant negative activity which is panoramic (i.e., has an effect of the same kind on multiple receptors) over two or more receptors. Further, the level or degree panoramic inhibition of biological activity may be or is substantially the same against said two or more receptors. Identification of panoramic capacity of any druggable ligand or DNL simply involves assaying the druggable ligand or DNL for inhibition of biological activity against the two or more receptors of interest.

The DNLs and DNL variants of the invention possess a number of uses. For example, the DNL variants of the present invention can be used to treat patients wherein dysregulation of cell signaling is implicated in the pathological process of disease (e.g. cancer, inflammation). Not only may the molecules of the present invention be administered as polypeptide based molecules, they may also be administered as nucleic acid molecules in the context of gene therapy. Furthermore, these molecules may be used in diagnostic applications as well as to further basic research.

Therapeutic Formulation and Delivery

The present invention also pertains to pharmaceutical compositions comprising the therapeutic DNL variants described herein. For instance, a DNL variant of the invention can be formulated with a pharmaceutically acceptable carrier or excipient to prepare a pharmaceutical composition. The carrier and composition can be sterile. The formulation should suit the mode of administration. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects such as nausea, dizziness, gastric upset and the like.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylase or starch, dextrose, magnesium stearate, talc, silicic acid, viscous paraffin, perfume oil, fatty acid esters, hydroxymethylcellulose, polyvinyl pyrrolidone, etc., as well as combinations thereof. In addition, carriers such as liposomes and microemulsions may be used. The DNL variants of the invention may also be covalently attached to a protein carrier such as albumin, or a polymer, such as polyethylene glycol so as to minimize premature clearing of the polypeptides. The pharmaceutical preparations can, if desired, be mixed with auxiliary agents, e.g. lubricants, preservatives, stabilizers, wetting agents, emulsifiers, salts for influencing osmotic pressure, buffers, coloring, flavoring and/or aromatic substances and the like that do not deleteriously react with the active agent in the composition (i.e., a polypeptide and/or nucleic acid molecule of the invention).

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrrolidone, sodium saccharine, cellulose, magnesium carbonate, etc.

Methods of introduction of these compositions include, but are not limited to, transdermal, intramuscular, intraperitoneal, intraocular, intravenous, subcutaneous, topical, oral, pulmonary and intranasal. In one embodiment, topical applications include those for treating conditions such as scarring, skin cancer, psoriasis, eczema.

Other suitable methods of introduction can also include gene therapy (as described below), rechargeable or biodegradable devices, particle acceleration devices (“gene guns”) and slow release polymeric devices. The pharmaceutical compositions of this invention can also be administered as part of a combination therapy with other DNLs or other compounds.

The DNL variants of the present invention can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentration in a hermetically sealed container such as an ampoule or sachette indicating the quantity of active compound (polypeptide and/or nucleic acid). Where the composition is to be administered by infusion, it can be dispensed with an infusion bottle containing sterile pharmaceutical grade water, saline or dextrose/water. Where the composition is administered by injection, an ampoule of sterile water for injection or saline can be provided so that the ingredients may be mixed prior to administration.

The DNL variants described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The DNL variants of the invention are administered in a therapeutically effective amount. The amount of DNL variant that will be therapeutically effective in the treatment of a particular disorder or conditions will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of the disease or condition, and should be decided according to the judgment of a practitioner and each patient's circumstances. Effective doses may be extrapolated from dose-response curves derived from in vitro or animal model test systems.

The present invention also pertains to methods of treatment (prophylactic, diagnostic, and/or therapeutic) for conditions characterized by dysregulation of cell signaling. A “condition characterized by dysregulation of cell signaling” is a condition in which the presence of a DNL variant of the invention is therapeutic. Such conditions include many types of cancer. Dysregulation of cell signaling has also been implicated in a variety of other disorders.

The term “treatment” as used herein, refers not only to ameliorating symptoms associated with the disease or condition, but also preventing or delaying the onset of the disease, and also lessening the severity or frequency of symptoms of the disease or condition. More than one DNL variant of the present invention can be used concurrently as a co-therapeutic treatment regimen, if desired. As used herein, a “co-therapeutic treatment regimen” means a treatment regimen wherein two therapeutic modalities are administered simultaneously, in either separate or combined formulations, or sequentially at different times separated by minutes, hours or days, but in some way act together to provide the desired therapeutic response. The DNL variants of the invention may also be used in conjunction with other therapeutic modalities that inhibit various aberrant activities of dysregulated cell signaling. Such additional therapeutic modalities include but are not limited to receptor specific antibodies, small molecule receptor inhibitors, traditional chemotherapeutic agents, and radiation treatment.

The therapeutic compound(s) of the present invention are administered in a therapeutically effective amount (i.e., an amount that is sufficient to treat the disease or condition, such as by ameliorating symptoms associated with the disease or condition, preventing or delaying the onset of the disease or condition, and/or also lessening the severity or frequency of symptoms of the disease or condition). The amount that will be therapeutically effective in the treatment of a particular individual's disease or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or condition, and should be decided according to the judgment of a practitioner and each patient's circumstances. Effective doses may be extrapolated from dose-response curves derived from in vitro or animal model test systems.

A therapeutically effective amount of a DNL variant of this invention is typically an amount of DNL variant such that when administered in a physiologically tolerable composition is sufficient to achieve a plasma concentration of from about 0.1 microgram (ug) per milliliter (ml) to about 100 ug/ml, preferably from about 1 ug/ml to about 10 ug/ml, and usually about 5 ug/ml. Stated differently, the dosage can vary from about 0.1 mg/kg to about 300 mg/kg, preferably from about 0.2 mg/kg to about 200 mg/kg, most preferably from about 0.5 mg/kg to about 20 mg/kg, in one or more dose administrations daily, for one or several days.

Dosages may also be based on the range of serum levels of the native ligand, for example, EGF (0.1-1 ng/ml) and/or relative to the affinity for the DNL. Using this starting point, compounds of the invention may be administered in doses up to ten-fold these measurements. For example, if the DNL affinity is 10 nM and the affinity for the native ligand is 1 nM, then the dosing range would be between about 10 ng/mL and about 100 ng/mL.

The present invention also contemplates the calculation of therapeutically effective amounts can be made relative to the starting ligand, for example EGF (about 1 nM). It is also understood that the doses of the compounds of the present invention may mirror those of other drugs such as Erbitux® (10.5 mg/kg) or Herceptin® (4 mg/kg).

The therapeutic compositions containing a DNL variant or a polypeptide of this invention may be administered via a unit dose. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle.

The therapeutic compounds of the present invention can be used either alone or in a pharmaceutical composition as described above. For example, the gene for a DNL variant of the present invention, either by itself or included within a vector, can be introduced into cells (either in vitro or in vivo) such that the cells produce the desired DNL polypeptide. If desired, cells that have been transfected with the nucleic acid molecule of the present invention can be introduced (or re-introduced) into an individual affected with the disease.

Gene Therapy

The therapeutic DNL variants of the present invention may also be used in the context of gene therapy. In the meaning of the invention, “gene therapy” is a form of treatment using natural or recombinantly engineered nucleic acid constructs, single gene sequences or complete gene or chromosome sections or encoded transcript regions, derivatives/modifications thereof, with the objective of a biologically based and selective inhibition or reversion of disease symptoms and/or the causal origin thereof.

For example, gene therapy may be effected using suitable vectors such as viral vectors or/and complex formation with lipids or dendrimers. Gene therapy may also proceed via packaging in protein coats. Furthermore, the polynucleotide can be fused or complexed with another molecule supporting the directed transport to the target site, uptake in and/or distribution inside a target cell. The kind of dosage and route of administration can be determined by the attending physician according to clinical requirements. As is familiar to those skilled in the art, the kind of dosage will depend on various factors, such as size, body surface, age, sex, or general health condition of the patient, but also on the particular agent being administered, the time period and type of administration and on other medications possibly administered in parallel, especially in a combination therapy.

The therapeutic DNL variants of the invention may also be contained within a kit. As such, the invention also relates to a kit comprising the therapeutic DNL variant and/or the pharmaceutical composition. Furthermore, the invention also relates to an array comprising the therapeutic DNL variants and/or the pharmaceutical composition.

Kits and arrays can be used in the diagnosis and/or therapy of diseases associated with the dysregulation of cell signaling. The invention also relates to the use of said therapeutic DNL variant, said kit, said array in the diagnosis, prophylaxis, reduction, therapy, follow-up and/or aftercare of diseases associated with dysregulation of cell signaling.

### EXAMPLES

### Example 1

### Methods and Reagents

Cloning and gene expression. The human epidermal growth factor gene (EGF) was synthesized chemically and ligated into the Pet-9a vector (Novagen) at the NdeI and BamHI cloning sites. The EGF gene contained the OmpA leader sequence followed by an N-terminal 6×-his tag (underlined) and a factor Xa cleavage site for future his-tag removal, (BOLDED: IEGR) if necessary, and corresponds to the following amino acid sequence:

| | |
|---|---|
| EGF gene clone | SEQ ID NO |

| | |
|---|---|
| MKKTAIAIAVALAGFATVAQAHHHHHHIEGRNSDSECPLSH | 1 |
| DGYCLHDGVCMYIEALDKYACNCWGYIGERCQYRDLKWWELR | |

This original clone, designated pMLPP1, was used as a basis for cloning all Pan HER ligand variants (including substitution, deletion, insertion and domain swap variants) using the QuickChange mutagenesis kit (Stratagene). For protein production the EGF plasmids were transformed into E. coli strain BL21 (DE3) pLysS (Novagen).

Production of Ligand Variants.

Single colonies were inoculated into shake flask cultures containing 15 ml LB+Km25+Cm30. After growth overnight, samples of culture were frozen for stocks, and for plasmid preps to confirm the identities of the EGF variant gene inserts. The remaining cultures were used to inoculate production cultures in Terrific Broth+Km25+Cm30. Cells were induced with 0.2 mM IPTG during early log phase, and the cultures were grown overnight. Culture supernatants were collected by centrifugation and production was confirmed by dot blot using the Mouse Western Breeze Chromogenic Immunodection System (Invitrogen cat#WB7103) with primary antibody: 1:1000 mouse anti-penta his antibody (Qiagen cat#34660).

EGF Protein Purification.

Three ml of Ni-NTA resin (Qiagen #30230) was used to pack 5 ml columns (Qiagen cat#34964) which were equilibrated with PBS pH 8.0. Culture supernatants were adjusted to pH 7.5-8.0 with 1N HCL before loading on columns. Columns were washed with PBS and PBS+10 mM imidazole; EGF variant proteins were eluted from columns with PBS+250 mM imidazole. Bradford protein assays were used to monitor protein concentrations.

Protein Concentrate and Buffer Exchange.

Column eluents were dialyzed in PBS at 4° C. with one buffer exchange, and then concentrated with 3000 MWCO Macrosep centrifuge devices (ISC# OD003C41). The final product was tested for protein concentration using the BCA method and for purity by SDS-PAGE.

### Example 2

### Design and Validation of Pan-HER Antagonists

Selection of Druggable Ligand and Domain Binding Optimization

Using native EGF as a starting druggable ligand, three N-terminal modification variants were created which improve binding. These modifications alter binding to HER3 with no effect on EGFR (HER1). The variants are listed in Table 2.

TABLE 2
EGF ligand variant

| EGF ligand | | |
|---|---|---|
| variant | N-terminal modification | SEQ ID NO |
| BiRegulin | Amino terminal residues | 2 |
| (BiR) | (NSDSE) are replaced with the | |
| | corresponding residues of | |
| | heregulin (SHLVK) | |
| WVS | Amino acids 2 and 3 are | 3 |
| | replaced with W and V | |
| | respectively, resulting in a | |
| | modified N-terminus sequence | |
| | of (NWVSE) | |
| T1E | Amino terminal residues | 4 |
| | (NSDSE) are replaced with | |
| | seven residues from TGF-α | |
| | (VVSHFND). N-terminal | |
| | domain swap with TGF-alpha. | |

To the modified druggable ligands of Table 2, further modifications were then made, which abrogate binding to Domain III in both EGFR and HER3. These modified ligands are listed in Table 3.

TABLE 3
Ligands modified to inhibit domain binding

| Ligand Variant | Background | Description | SEQ ID NO |
|---|---|---|---|
| wvs-R41DL47G | WVS | Amino acid R at position | 5 |
| | | 41 replaced by D; amino | |
| | | acid L at position 47 | |
| | | replaced by G | |
| wvs-R41D | WVS | Amino acid R at position | 6 |
| | | 41 replaced by D | |
| wvs-L47G | WVS | Amino acid L at position | 7 |
| | | 47 replaced by G | |

### Example 3

### Use of Phage Display Vectors to Produce and Assay Pan-HER Antagonists

Library Construction and Phage Panning

Two libraries were constructed using the Kunkel procedure. Random clones were sequenced from each library and it was calculated that each nucleic acid variant was represented between 500 and 1000 times and each amino acid sequence variant was represented between 10⁴-10⁵ times.

As a starting point, the libraries were constructed to contain the modified agonists and antagonists or combinations thereof from Example 2 in addition to alterations in the B-loop of EGF, which is known to be critical for binding to Domain I of the EGF receptor, at either residues 21-25 or 26-30. A selection of members from the libraries are shown in Tables 4-7.

TABLE 4

Library PD1B: Residues 21-35: first half of
B-loop Preamplification

| | | | |
|---|---|---|---|
| Ligand | | Amino acid | SEQ ID |
| Variant | Codon Sequence | sequence | NO |

| | | | |
|---|---|---|---|
| wvs-R41DL47G | ATG TAT ATT GAA GCG | MYIEA | 5 |
| PD1B-25 | CGT GCG CTA GCG AGG | RAVAR | 8 |
| PD1B-26 | AAG AAT TAT AAT GAG | KNYNE | 9 |
| PD1B-29 | TAT ATG AAG GGG GGG | YAKGG | 10 |
| PD1B-34 | GGT GGG GGG AAG GCG | GGGKA | 11 |
| PD1B-37 | GGT GGG TCG AAG GGG | GGSKG | 12 |
| PD1B-40 | AGG GAG AGG ACG GGT | RERTG | 13 |
| PD1B-33 | CCG CGG ACT GCT CCG | PRTAP | 14 |

TABLE 5

Library PD1B: Residues 21-35: first half of
B-loop Amplified

| | | | |
|---|---|---|---|
| Ligand | | Amino acid | SEQ ID |
| Variant | Codon Sequence | sequence | NO |

| | | | |
|---|---|---|---|
| wvs-R41DL47G | ATG TAT ATT GAA GCG | MYIEA | 5 |
| PD1B-41 | ACG ACG CAG ACG CCG | TTQTP | 15 |
| PD1B-42 | ACG AAT AAG GAG AGG | TNKER | 16 |
| PD1B-43 | TCG GGG AGG CCG ACG | SGRPT | 17 |
| PD1B-44 | ATG GGT ATG GGG CGG | MGMGR | 18 |
| PD1B-45 | ATG GGG AGT TGC GGG | MGSSG | 19 |
| PD1B-46 | ACG ACG AAT AAG GCG | TTNKA | 20 |
| PD1B-47 | AAG CCG GAG AAG CAG | KPEKQ | 21 |
| PD1B-50 | GAT AAT CCG ATG CGT | DNPMR | 22 |
| PD1B-52 | GGG CCG CAG GCT CCT | GPQAP | 23 |

TABLE 6

Library PD2B: Residues 26-30: second half of
B-loop Preamplification

| | | | |
|---|---|---|---|
| Ligand | | Amino acid | SEQ ID |
| Variant | Codon Sequence | sequence | NO |

| | | | |
|---|---|---|---|
| wvs-R41DL47G | CTG GAT AAA TAT GCG | LDKYA | 5 |
| PD2B-37 | CAT CCC AAG TCT TAT | HPKSY | 24 |
| PD2B-38 | ACT CCT TCT TAT TTG | TPSYL | 25 |
| PD2B-39 | AAT CGC GAG AAG ACT | NREKT | 26 |
| PD2B-40 | AGT AAG CGT CAG CCG | SKRQP | 27 |
| PD2B-41 | CAG ATT AAG CTT CTG | QIKLL | 28 |
| PD2B-44 | GGG ACT AAG CAT CGG | GTKHR | 29 |
| PD2B-45 | ATT AGC TTG CGG CCT | ISLRS | 30 |
| PD2B-47 | GGG ACT GCG CGT CCT | GTARG | 31 |
| PD2B-48 | GAG AAT AAG CGT CCT | ENKRR | 32 |

TABLE 7

Library PD2B: Residues 26-30: second half of
B-loop Amplified

| | | | |
|---|---|---|---|
| Ligand | | Amino acid | SEQ ID |
| Variant | Codon Sequence | sequence | NO |

| | | | |
|---|---|---|---|
| wvs-R41DL47G | CTG GAT AAA TAT GCT | LDKYA | 5 |
| PD2B-49 | TAT GGT AAT ACT ACG | YGNTT | 33 |
| PD2B-50 | GAT CGG TCT CTT ACG | DRSLT | 34 |
| PD2B-51 | TCG CAT GGG CAG GAG | SHGQE | 35 |
| PD2B-52 | CAT ATT GCT GGT GCT | QIAGA | 36 |
| PD2B-53 | CCT AAT CCT AGT CCG | PNPSP | 37 |
| PD2B-54 | GGT AAG TCG AGT AAG | GKSMK | 38 |
| PD2B-55 | CAG CCG CAT TTG TCT | QPHLS | 39 |
| PD2B-56 | CCT CAC GCG TCT CTT | PHASL | 40 |
| PD1B-59 | CAG ATG CAG TCG CGT | QMQSR | 41 |

Phage panning was performed according to the teachings Smith and Petrenko (Smith, G. P. and V. A. Petrenko. 1997. Phage Display. Chem. Rev. 97:391-410.) Briefly, genes for the three pan-HER agonists (T1E, WVS, and BiR), were selected for study. These genes coding domain binding optimized druggable ligands, were cloned into the pentavalent M13 phage display system (New England Biolabs) along with mutations that reduce binding to HER receptor domains (e.g. R41D and L47G) using the Kpn I and Eag I restriction sites of the M13KE phage vector for expression as an N-terminus-fusion with the pIII coat protein of the M13 phage.

All five copies of pIII should display the cloned protein. To produce phage, the vector with insert was transformed into electrocompetent E. coli 10 GF'. Transformation outgrowth was used to infect E. coli and infected cells were plated on LB+tet20+xgal+IPTG. Blue plaques resulting from the infection were amplified and plasmid DNA was sequenced to verify the identity of the insert. Phage were amplified by infecting E. coli in LB culture, and cells were removed by centrifugation. Phage were harvested by PEG precipitation. These phage were used to measure biological activity by stimulation of HER receptor dependent cell proliferation.

Phage ELISA for Analysis of Binding Affinity

A431 cells for EGFR binding or T47D cells for HER3 binding were grown as monolayers in tissue culture flasks in media containing fetal bovine serum. Cells were trypsinized, neutralized with growth medium, washed twice with DPBS and resuspended in ice-cold PBS-Glu-T. 10₅ cells were transferred to 96 well plates and incubated on ice for 1 hour in the presence of varied concentrations of phage. Cells were centrifuged and washed 5× with PBS-T then incubated for one hour at room temperature with anti-M13 pVIII coat protein antibody conjugated with horseradish peroxidase (HRP). Cells again centrifuged and washed 5× with PGS-T. Color developed with TMP followed by H₂SO₄. Cells pelleted and supernatant transferred to optically transparent plate for measurement of absorbance at 450 nm.

Theoretical estimates were also performed. The results are shown in Table 8. “N.D.” indicates not determined. EC50 is the concentration of phage necessary for a 50% stimulation of cell proliferation. From the binding curves it is evident that ligand binding was completely abrogated by the wvs-R41□L47G ligand variant.

TABLE 8
Binding affinity of ligand variants: T47D cells

| Ligand | Estimated binding (EC50) | Calculated binding (EC50) |
|---|---|---|
| Variant | phage titer/mL | phage titer/mL |
| WVS | 9 × 10⁸ | 9.8 × 10⁹ |
| T1E | 1 × 10¹⁰ | ND |
| wvs-R41D | >1 × 10¹² | ND |
| wvs- | ND | 2.7 × 10²⁰ |
| R41DL47G | | |

Phage particles displaying ligand variants were evaluated for binding affinity to the HER3 receptor in A431 whole cell suspensions by measuring absorbance at Abs450. The results are shown in Table 9. From the binding curves it is evident that ligand binding was totally abrogated by the wvs-R41DL47G and wvs-R41D ligand variants.

TABLE 9
Binding affinity of ligand variants: A431 cells

| | Calculated binding (EC50) |
|---|---|
| Ligand Variant | phage titer/mL |
| WVS | 6.6 × 10⁸ |
| wvs-R41D | 5.8 × 10¹¹ |
| wvs-L47G | 6.6 × 10¹⁰ |
| wvs-R41DL47G | 4.8 × 10¹² |

Binding of phage to receptor was analyzed via phage ELISA. The results indicate the ability to distinguish among high affinity binders (WVS) low affinity binders (RL, ablated at one binding face) and non-binders (ablated at both binding faces). EGF has also been used to compete with WVS phage, to confirm the receptor-specificity of binding phage.

Cell Lines

HER5 Cells

The HER5 cell line, a murine fibroblast line (derived from the NR-6 line) that has been stably transfected to express the human EGF receptor was provided by Dr. M. C. Hung (MD Anderson Cancer Center).

MCF-7 Cells

MCF-7 cells were obtained from the American Type Culture Collection (ATCC). Stock cultures of MCF-7 were maintained in Eagle's MEM supplemented with 1% ITS-X (Invitrogen) and 10% fetal bovine serum.

T47-D Cells

Human ductal carcinoma cells were obtained from ATCC. They were maintained in RPMI 1640 medium with 2 mM L-glutamine adjusted to contain 1.5 g/L sodium bicarbonate, 4.5 g/L glucose, 10 mM HEPES, and 1.0 mM sodium pyruvate and supplemented with 0.2 Units/ml bovine insulin, 90%; fetal bovine serum, 10%.

### Example 4

### Biological Activity of Phage-Fusions

It was unexpectedly discovered herein that, not only could phage particles be used to measure binding affinity, but that these same particles displaying the ligand variants of the invention could also be used directly in assays to determine biologic activity.

Phage-fusion particles displaying ligand variants were evaluated for their ability to stimulate cell proliferation in the cell proliferation assay described herein in both an EGF dependent cell line, HER5 and a heregulin dependent cell line, MCF-7. The data are summarized in Tables 10 and 11.

TABLE 10
Cell Proliferation: HER5 cells

| | Calculated cell proliferation |
|---|---|
| Ligand Variant | (EC50; picomolar) |
| EGF (purified protein) | 1150 |
| BiR (phage) | 3.2 |
| T1E (phage) | 2.6 |
| WVS (phage) | 4.9 |
| T1ER41D (phage) | 2800 |

TABLE 11
Cell Proliferation: MCF-7

| | Calculated cell proliferation |
|---|---|
| Ligand Variant | (EC50; picomolar) |
| Heregulin (purified protein) | 6151 |
| T1E (phage) | 3052 |
| WVS (phage) | 237 |
| T1ER41D (phage) | >10⁶ |

It is known that HER5 cells can be stimulated by EGF and BiR but not by HRG, while MCF-7 cells can be stimulated by BiR and HRG but not by EGF. It has also previously been demonstrated using isolated ligand variants that the pan-HER agonists T1E, WVS, and BiR, are all capable of stimulating cell proliferation in EGFR-dependent HER5 cells while the weak binding mutant TLER41D has greatly attenuated activity and that MCF-7's are stimulated most effectively by WVS and not at all by BiR.

Here it is demonstrated that the EGF variant WVS was not only able to stimulate cell proliferation in the engineered mouse fibroblast cell line HER5 but was more potent than hEGF itself. It should be noted that WVS and T1E phage are more potent than the purified protein ligands of EGFR and HER3. This is due to the pentavalent state of the phage fusions which results in increased apparent affinity due to avidity effects.

This effect is a function of the EGF variant in display; inactive variants or no insert controls do not stimulate cell proliferation. And this effect is not limited to this cell line or growth factor. Variants of heregulin-β displayed as coat protein fusions also stimulate the growth of HER2/HER3 dependent cell lines. Now it is possible to screen for antagonist properties in the phage themselves and work with isolated protein only as a confirmatory test.

Cell Proliferation Stimulation Assays

HER5 Cells

Stock cultures of HER5 were propagated in D-MEM/F12 medium containing 10% fetal bovine serum, 100 units/ml of penicillin and 100 ug/ml of streptomycin in a water-jacketed incubator at 37° C. in a humidified 5% CO₂ atmosphere.

For HER5 proliferation assays, the cells were changed into DMEM/F12 without serum for 24 hours. Cells were then trypsinized and suspended at 1E5 cells/ml. Serial dilutions of EGF (PeproTech, Rocky Hill, N.J.), and HER ligand polypeptide variants were prepared in serum-free DMEM/F12 at 2-fold the final concentration and plated into the wells of 96-well plates. Fifty microliters of cell suspension (5000 cells) were added to appropriate wells bringing the total volume to 100 ul at the desired concentrations. Plates were incubated for a 48 hour proliferation period. Cell proliferation was determined by addition of 10 ul/well of WST-1 Cell Proliferation Reagent (Roche Applied Sciences, Indianapolis, Ind.) for the last three hours of the proliferation period. WST-1 is a tetrazolium salt that is cleaved to formazan dye by mitochondrial dehydrogenases in viable cells. The amount of formazan was measured at 450 nm using a microplate reader (Dynex Technologies) with MRX Revelation software.

MCF-7 Cells

For proliferation assays, MCF-7 cells were transferred to serum-free medium (SFM) for 24 hours and then trypsinized and suspended at 1E5 cells/mL in SFM. Fifty microliters of cell suspension (5000 cells) were plated per well in 96 well microtiter plates. Serial dilutions of HER ligands or mutant proteins were prepared at twice the final concentration in SFM and 50 ul was added to wells, bringing the final volume to 100 ul at the desired final concentration. Plates were incubated for 72 hours at 37 C in a humidified 5% CO₂ atmosphere. Cell proliferation was determined by addition of 10 ul/well of WST-1 Cell Proliferation Reagent (Roche Applied Sciences, Indianapolis, Ind.) for the last three hours of the proliferation period.

We have demonstrated that Pan-HER agonists T1E, WvS, and BiR, are all capable of stimulating cell proliferation in EGFR-dependent HER5 cells while the weak binding mutant TLER41D has greatly attenuated activity. The HER2/HER3 dependent cell line MCF-7 is stimulated most effectively by WvS and not at all by BiR. We conclude therefore that WvS and T1E act as Pan-HER agonists, capable of binding to and activating EGFR, HER3, and HER4.

### Example 5

### Domain Swap to Alter Selectivity

A domain swap was undertaken within the B-loop of EGF (residues 21-30). This swap was expected to further enhance ligand variant binding, particularly to Domain I of the EGF receptor and HER3 receptor. The first half of the B-loop, (amino acid residues 21-25), and the second half of the B-loop (residues 26-30) were rationally redesigned to produce the variants in Table 12. The variants, D4, D4-2 and E8 were all prepared on the WVS background.

The phage fusion ligand variants were then evaluated for binding using the assay described herein in both A431 cells (to investigate EGFR binding) and T47D cells (to investigate HER3 receptor binding) and EC50s were calculated. Binding data are shown in Tables 13 and 14.

TABLE 12

Ligand variants

| | | | |
|---|---|---|---|
| Ligand | B-loop | First half/ | |
| Variant | Sequence | second half | SEQ ID |

| | | | |
|---|---|---|---|
| WVS | MYIEALDKYA | Wild type/Wild type | 3 |
| WVS-R41DL47G | MYIEALDKYA | Wild type/Wild type | 5 |
| D4 | MYIEAYRVKT | Wild type/YRVKT | 42 |
| D4-2 | YRVKTLDKYA | YRVKT/Wild type | 43 |
| E8 | MYIEATKYRG | Wild type/TKYRG | 44 |

TABLE 13
Binding of ligand variants (First half loop)

| | Calculated binding | Calculated binding |
|---|---|---|
| | (EC50) in A431 cells | (EC50) in T47D cells |
| Ligand Variant | phage titer/mL | phage titer/mL |
| WVS | 1.9 × 10⁹ | 7.5 × 10⁹ |
| WVS-R41DL47G | 2.8 × 10¹⁰ | 1.1 × 10¹² |
| D4-2 | 3.6 × 10¹⁰ | 8.6 × 10¹⁰ |

The D4-2 ligand variant having a half-loop modification (YRVKT) in the first half was determined to bind only the HER3 receptor and is therefore not panoramic to multiple EGF receptors. Consequently, D4-2 is a HER3 specific antagonist.

TABLE 14
Binding of ligand variants (Second half loop)

| | Calculated binding | Calculated binding |
|---|---|---|
| | (EC50) in A431 cells | (EC50) in T47D cells |
| Ligand Variant | phage titer/mL | phage titer/mL |
| WVS | 1.4 × 10⁹ | 7.6 × 10⁹ |
| WVS-R41DL47G | 4.0 × 10¹⁰ | 2.6 × 10¹¹ |
| D4 | 3.9 × 10⁹ | 2.9 × 10¹⁰ |
| E8 | 5.2 × 10⁹ | 3.1 × 10¹⁰ |

Binding curves and EC50 calculations show that the D4 and E8 variants have intermediate binding properties for both receptors between that of the WVS variant and the WVS-R41DL47G variant.

Together these data indicate that the half-loop modification can yield ligands with improved binding properties (compare WVS-R41DL47G having an wild type B-loop with D4 having a modified second half loop). Furthermore, it is demonstrated that by moving the half loop modification found to improve Domain I binding in D4 from residues 26-30 to residues 21-25 in the B-loop producing variant D4-2, binding can be selectively enhanced for one receptor over another. It is also contemplated that using this method, receptor binding may be a titratable property in the optimization of therapeutic ligands.

Understanding that in certain cases, it will be important to design ligand variant which are selective for one receptor over another, the following examples expand on the design methods herein to produce anticancer ligands which target the IGF-IR receptor but not the IR.

Examples 5-10 relate to the design and validation of DNLs and anticancer ligands (ACLs) using the IGF-IR/IR signaling system.

### Example 6

### Design and Validation of DNLs: IGF-IR Selective Ligands

Production and Analysis of IGF-I.

High-level production of IGF-I has been achieved (by others) in a variety of cloning hosts such as E. coli, Staphylococcus aureus and yeast (Forsberg, G., et al., (1990) Biochem. J. 271:357-363; Moks, T., et al., (1987) Biochemistry. 26:5239-5244).

IGF-I is being manufactured commercially by at least two companies (Tercica and Insmed) for use in clinical trials to treat IGF-I Deficiency Disorder.

In the cloning studies herein, the IGF-I gene was constructed using overlapping oligos and ligated it into the pET-9a vector (Novagen) at the NdeI and BamHI cloning sites. The IGF-I gene was fused to the OmpA leader sequence for export to the periplasm and also contained sequence for an N-terminal his-tag with a factor Xa cleavage site. The resultant clone corresponds to the following amino acid sequence:

| IGF-I gene clone | SEQ ID NO |
|---|---|
| MKKTAIAIAVALAGFATVAQAHHHHHHIEGRGPETLCGAEL | 45 |
| VDALOFVCGDKGFYFNKPTGYGSSSRRAPOTGIVDECCFRS | |
| CDLRRLEMYCAPLKPAKSA | |

The resulting plasmid was transformed into E. coli strain BL21 (DE3) pLysS (Novagen) and protein production was confirmed by dot blot using the Mouse Western Breeze Chromogenic Immunodection System (Invitrogen) with primary antibody: mouse anti-penta his antibody (Qiagen). The IGF-I produced in E. coli was purified by Ni-IMAC column chromatography and confirmed in assays for ability to stimulate cell proliferation on two sensitive cell lines (MCF-7 and HT-29) with cell density monitored by reaction with WST-1 Cell Proliferation Reagent (Roche Applied Sciences). The his-tagged material appeared to have slightly reduced activity compared to commercial preparations (Pepro), but the his-tag can be removed and the resultant cleavage product (purified by size exclusion chromatography) is indistinguishable from the commercial material.

The standard cell line for evaluating compounds that interfere with IGF-I dependent growth is the breast cancer cell line MCF-7. This cell line expresses over 43,000 copies of IGF-IR per cell, but the dynamic range of response is rather low relative to that of the colon cancer cell line HT-29.

It is understood in the art that other cell lines may be used to screen. For example cell lines with superior response to IGF-I as well as cell lines that do not respond to IGF-I may be used as negative controls. The mouse fibroblast cell line NIH/3T3, is an example of the latter category.

### Example 7

### Evolutionary Trace Analysis of IGF-I

Evolutionary Trace (“ET”) is an algorithm that compares and contrasts related DNA sequences (Lichtarge, O., et al., (1996) J Mol Biol 257, 342-58; Sowa, M. E., et al., (2000) Proc Natl Acad Sci USA 97, 1483-8; Lichtarge, O. and M. E. Sowa. (2002) Curr Opin Struct Biol 12, 21-7). It identifies conserved amino acid residues but more importantly residues that are unique to a particular sub-set of proteins, and ultimately to a particular protein. When these “Trace Residues” are mapped onto the surface of proteins, they frequently describe “Trace Clusters”. In about 85% of the reported cases (out of hundreds tested) these trace clusters map to functional sites. Insulin and its related family of proteins represent a group of structurally related polypeptides whose functions have diverged (Lu, C., et al., (2005) Pediatr Res. 57:70R-73R). There are 167 protein sequences in the public databases that share at least 15% homology with human IGF-I.

The ET analysis begins by preparing a dendogram showing related proteins, including various insulins, other IGF-Is and IGF-IIs. Particular amino acids are identified as points of divergence between related proteins, and statistically ranked. The lowest rank (most important) trace residues were mapped to the same crystal structure used in the Denley summary (Denley et al., (2005) Cytokine Growth Factors Reviews 16:421-439). All the low rank residues mapped to a single well defined region. None of the low rank residues mapped to the other face of the molecule. These are the residues that set the Insulin/IGF-I family of proteins apart from others, and define a common domain and clearly map out one binding face of the protein. The trace analysis corroborates published mutational studies, implicating four very important residues in the “common” domain (Domains A and B): F16, F23, Y24 and V44.

The binding surface characterized by these residue can be ablated because it will eliminate the binding of our antagonist to Binding Face 1 of both IGF-IR and IR.

The second binding surface of IGF-I is also well defined by the mutational analysis and is made up of residues in Domains C and D. It is known that alanine substitutions in these domains (which remove important functional residues) decrease affinity for IGF-IR and increase affinity for IR (Zhang, W., et al., (1994) J Biol Chem. 269:10609-10613). Thus, these regions are responsible for the differences in binding to the two receptors. Therefore this region can be engineered to enhance the affinity for IGF-IR Binding Face 2. Such modifications are likely to reduce binding affinity to IR.

Further support for the hypothesis that IGF-I is a divalent ligand comes from the structural and sequence similarities between the IGF-I/IGF-IR system and the EGF/EGFR system. It is very likely that IGF-I plays a similar role to EGF in stabilizing the close association of two normally distant domains of the receptor.

### Example 8

### Direct, Non-Radioactive Binding Assay

A non-radioactive method to measure binding of EGF to EGFR using biotinylated EGF and horseradish peroxidase bound to streptavidin (De Wit, R., et al., (2000) J Biomol Screen. 5:133-140) has been modified herein. Rather than follow displacement of ¹²⁵I-labeled EGF, oxidation of Ultra ELISA TMB (Pierce) is followed. This assay yields binding constants comparable to published data.

Additional Studies

Further assay development can be performed to measure competition of IGF-I variants with biotinylated wild-type IGF-I.

### Example 9

### Point Mutations of IGF-I that Ablate the Common (IR and IGF-IR) Binding Surface. (Surface 1)

According to published reports and our ET analysis, four very important residues in the “common” domain are F16, F23, Y24 and V44. Based on the crystal structure data (Vajdos, F. F., et al., (2001) Biochemistry 40:110221102-9) these residues are all found on one surface of IGF-I. The point mutants F16A, F23G, Y24L, and V44M all demonstrated nearly two orders of magnitude lower binding to IGF-IR. In addition, all variants save F16A were also tested for binding to IR and have significantly reduced affinities for this receptor as well.

Additional Studies

The wild-type IGF-I gene has been cloned into the M13 phage vector to generate a fusion protein with the minor coat protein pIII (Ph.D. Peptide Display Cloning System, New England BioLabs). While there are no published accounts of phage display and panning of IGF-I in the scientific literature, U.S. Pat. No. 6,403,764, incorporated herein in its entirety, describes the approach in detail. Ballinger, M. D., et al., has disclosed the use of phage display to identify IGF-I variants with improved affinity to BP1 and BP3 (Ballinger, M. D., et al., (1998) J Biol. Chem. 273:11675-11684).

According to the present invention, the “pentavalent” M13 (rather than the monovalent) will be used to take advantage of avidity effects. The monovalent system is more appropriate when starting with high (nM) affinity. Since it is intend to start with attenuated binders (after ablating binding at Binding surface 1), the M13 pentavalent system is more appropriate because it has a better dynamic range at the binding affinities (μM) we expect to encounter. The inventors have observed this avidity effect with pentavalent display of EGF variants, and shown that they can distinguish between high affinity phage (1 nM), low affinity phage (10 uM) and parental phage with no inserts (non-binders).

The QuikChange® system from Stratagene can then be used to make the permutations of the mutations listed above. These variants, along with wild-type, can be produced in phage and tested for binding to IGF-IR and IR in phage ELISAs using immobilized IGF-IR ectodomain (R&D BioScience) and anti-pVIII antibody (New England Biolabs). Phage with no inserts as a negative control to define the limits of non-specific binding.

Phage ELISAs can also be performed in competitive mode with IGF-I to confirm specific binding. Some of the mutants constructed are likely to fold incorrectly, especially if the target residues are involved in structural integrity rather than binding interactions. It is expected that these variants will bind with an affinity close to the non-insert negative control. The goal is to identify a variant with the lowest measurable binding (somewhere in the range of 1-10 uM). Low but measurable binding is desirable as it is in the appropriate range to detect improvements in subsequent panning experiments. None of the reported single or combination mutants restricted to Domains A and B reduced binding by more than two orders of magnitude (Denley, A., et al., (2005) Cytokine Growth Factors Reviews 16:421-439), and it is possible that this is the limit as long as binding via Domain C and D is intact.

It has recently been observed by inventors that EGFR variants displayed on pentavalent phage retain the ability to stimulate cell proliferation in EGF-dependent cell lines and this observation has greatly accelerated this research program because it is now possible to screen variants for agonist properties without a need to reclone into expression systems for production and purification. It is expected that testing phage displaying agonist variants of IGF-IR for their ability to stimulate cell proliferation will also be successful because IGF-I function has been retained in genetic fusions with proteins ten times the size of the growth factor (Sandoval, C., H. et al., (2002) Protein Eng. 15:413-418). If successful, this result will allow the development of a phage agonist assay to relate the binding affinity of the phage to the potency in stimulating cell proliferation.

### Example 10

### Phage Display to Identify Domain C Variants with Improved Binding Via the Second Binding Site on IGF-IR

Domain C of IGF-I consists of residues 30-41; Domain D of residues 63-70. Though phage display can be used to sort through very large libraries, the complete randomization of only Domain C would lead to library size of 4×10¹⁵ amino acid sequence variants, far beyond the reach of this technology. Construction of libraries with six residues randomized, while still technically challenging, will only contain 6×10⁷ protein sequences, and randomized patches of this size can still lead to affinity improvements of more than an order of magnitude (Ballinger, M. D., et al., (1998) J Biol. Chem. 273:11675-11684).

To identify improved binders, residues 32-39 and 64-69 can be randomized, as these are centered around amino acids that are critical for binding (Zhang, W., et al., (1994) J Biol Chem. 269:10609-10613). The sites, R37R38 of Domain C and K65K68 of Domain D are particularly interesting.

Using the minimal binding variant identified in the phage panning studies in its M13 vector as a starting template the two regions can be partially randomized in individual libraries using Kunkel mutagenesis (Kunkel, T. A., et al., (1987) Methods Enzymol. 154:367-382). (The first two bases of each codon will be randomized, the third held to G or T, reducing the number of genetic variants and eliminating truncations due to stop codons.) The libraries can be electroporated into E. coli XL1-Blue (Stratagene). Random transformants can be sequenced to determine the percentage of variants and enough electroporations will be carried out to yield 3× the number of genetic variants in the theoretical library to achieve 90% confidence level that each variant is represented by at least one copy.

The phage plasmids can then be recovered and transformed into E. coli strain ER2738 (New England BioLabs) optimized for phage amplification. The libraries can be combined to find which domain individually can contribute to the largest increase in binding affinity. Although it is expected that the Domain A and B mutations will greatly attenuate binding to IR, a subtractive binding step can be used to eliminate strong binders. The method described in U.S. Pat. No. 6,403,764 can be used to accomplish this step, incubating the amplified phage library with immobilized IR ectodomain (R&D Bioscience). Phage-fusions that do not bind to IR will be removed in the supernatant and panned against IGF-IR, using immobilized IGF-IR ectodomain (R&D BioSciences).

Unbound phage can then be eliminated with buffer washes, and bound phage will be eluted with 0.2 M glycine-HCl (pH 2.2), 1 mg/ml BSA. Clones can be isolated from an eluant sample for sequencing and the remainder will be used for subsequent rounds of amplification and panning.

After four rounds of panning, binding phage can be isolated and binding affinities relative to the starting mutant and wild-type phage can be determined using phage ELISA with both IGF-IR and IR. Phage with improved affinity towards IGF-IR and lower affinity towards IR can then be tested for binding to IGF-IR specifically in competitive assays with IGF-I. Those phage can then be tested in a phage agonist assay to demonstrate progress towards decoupling binding with receptor activation.

It is expected that this will increase the affinity/potency ratio (Kd/EC50).

If none of the improved binders show antagonist characteristics, it may be necessary to further attenuate binding at Binding surface 1 with additional mutations in Domains A and B through the process of re-design.

When phage with the antagonist phenotype are identified, their affinity can be compared with that of wild-type IGF-IR phage. It is possible that none of the best binders will bind to IGF-IR with the affinity of IGF-I (<1 nM) because the subdomains being randomized are only six residues long. If that is the case, best binders may not be combined from each library because these sorts of interactions are rarely additive, especially in such a small protein. Rather, the clone with the best characteristics can be used as the next starting point and randomize the remaining subdomain for new rounds of panning. While two rounds of iteration may be sufficient, subsequent rounds are contemplated. At the end of this process, it is expected that 5-10 antagonist phage with affinities <10 nM will be identified. These will be carried forward into the next stage for confirmation of antagonist properties.

### Example 11

### Confirmation of Antagonist Properties

The hits identified above can then be cloned into the expression vector pET-9a (Novagen) and expressed in E. coli strain BL21 (DE3) pLysS (Novagen). This expression system has been modified to include the OmpA leader sequence followed by an N-terminal 6×-his tag and a factor Xa cleavage site for future his-tag removal. The proteins can be produced in shake flask and purified with Ni IMAC chromatography (Qiagen). This system has successfully been used to produce wild-type IGF-I. Any variants that do not express well or form inclusion bodies can be discarded. If necessary panning eluents may be returned to identify variants with acceptable production characteristics.

Variants can be tested for the ability to compete with biotinylated IGF-I and biotinylated insulin for binding to IGF-IR and IR. These tests will confirm that the variant protein has an affinity for IGF-IR within an order of magnitude of natural IGF-I and at least three orders of magnitude lower binding to IR (maintaining the same relative affinities of IGF-I). The binding of the variants with the highest affinity to IGF-IR in this assay will be confirmed by Biacore Surface Plasmon Resonance analysis, binding the variant protein to the chip and measuring changes in plasmon resonance by incubation with IGF-IR ectodomain (Denley A., et al., (2005) Mol. Endocrinol. 19:711-721). This analysis can optionally be carried out on a contract basis at the University of Texas Medical School Molecular Genetics Core Facility.

Using the methods herein or standard protein production methods enough protein can be produced for more detailed analysis. These purified proteins can be used in the following assays:

Inhibition of MCF-7 Cell Proliferation:

MCF-7 cells are grown as in the proliferation assay but are then transferred to either serum-free medium containing a level of IGF-I sufficient to stimulate significant growth or to medium with serum. Varied levels of IGF-I variants are then added to wells and cell proliferation is allowed to proceed as before. Interference with IGF-I stimulation is determined by reduction of absorbance at increasing concentrations of variant.

Inhibition of IGF-IR Autophosphorylation:

Binding of IGF-I to IGF-IR leads to activation and autophosphorylation of the receptor kinase domain. MCF-7 cells treated with IGF-I in the presence and absence of our variants are used to generate lysates. The lysates are first normalized for levels of IGF-IR using total IGF-IR ELISAs (R&D Systems) Autophosphorylation is then monitored with phosphor-IGF-IR ELISAs (R&D Systems). It is expected that the variants will interfere with IGF-1-stimulated phosphorylation of tyrosine 1131. The non-specific kinase inhibitor staurosporine (Sigma) and the EGFR-specific kinase inhibitor AG1478 (Sigma) are used as positive and negative controls.

Inhibition of IR Activity:

It can be demonstrated that the compounds of the invention are unable to interfere with IR-related activities by using McA-RH7777 rat hepatoma cells (ATCC) to demonstrate insulin-dependent IR autophosphorylation as well as phosphorylation of insulin receptor substrate (Hansson, P. K., et al., (2004) Biochim Biophys Acta. 1684:54-62) using western blots as described above.

The level of IR can be normalized using westerns with Anti-Insulin Receptor, β subunit (Upstate) and level of autophosphorylation can be determined using the phospho-IR ELISA kit (R&D Systems). No interference of IR-related phosphorylation with our variants at physiologically relevant concentrations is expected.

Cellular Toxicity Screening:

Proliferation assays are known in the art. These can be performed with a battery of non-IGF-I responsive cell lines including the mouse fibroblast NIH-3T3 and the human cancer cell lines CaLU-1 and SK-BR-3 to screen for general toxicity.

Phage ELISA for Analysis of Binding Affinity

A solid phase ELISA will be used for the analysis of IGF-I variant phage binding to IGF-IR. A capture antibody (mouse monoclonal antibody clone JBW902 (Upstate) with specificity for the kinase domain of IGF-IR) will be used to properly align the IGF-IR(R&D Systems). The plates will be blocked with PBS containing 1% BSA and then incubated with phage in varied concentrations. After several washes with PBS containing 0.1% Tween-20, the bound phage will be detected with anti-M13 pVIII coat protein antibody conjugated with HRP. Color will be developed with TMP followed by H₂SO₄ and absorbance at 450 nm will be measured.

Cell Proliferation Assay

Cells were grown in complete medium and then serum starved. Cells were incubated with purified growth factor or with phage for 48-72 hours. Cell proliferation was determined by addition of 10 ul/well of WST-1 Cell Proliferation Reagent (Roche Applied Sciences, Indianapolis, Ind.) for the last three hours of the proliferation period. WST-1 is a tetrazolium salt that is cleaved to formazan dye by mitochondrial dehydrogenases in viable cells. The amount of formazan was measured at 450 nm using a microplate reader (Dynex Technologies) with MRX Revelation software.

Cell Lines

HT-29

Human colorectal carcinoma cells were obtained from ATCC. They were cultivated in McCoy's 5a medium (modified) with 1.5 mM L-glutamine adjusted to contain 2.2 g/L sodium bicarbonate, 90%; fetal bovine serum, 10%.

NIH-3 T3

Mouse fibroblast cells were obtained from ATCC. They were cultivated in Dulbecco's modified Eagle's medium with 4 mM L-glutamine adjusted to contain 1.5 g/L sodium bicarbonate and 4.5 g/L glucose, 90%; bovine calf serum, 10%.

CaLU-1

Human lung epidermoid carcinoma cells were obtained from ATCC. They were cultivated in McCoy's 5a medium with 1.5 mM L-glutamine, 90%; fetal bovine serum, 10%.

SK-BR-3

Human breast cancer cells were obtained from ATCC. They were cultivated in McCoy's 5a medium (modified) with 1.5 mM L-glutamine adjusted to contain 2.2 g/L sodium bicarbonate, 90%; fetal bovine serum, 10%.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method for designing therapeutic dominant negative ligands (DNLs) comprising;
a) selecting a druggable ligand from the group consisting of a known receptor ligand and a polypeptide sequence designed to function as a druggable ligand, wherein the known or predicted structure of the druggable ligand presents or contains two or more receptor binding surfaces,
b) performing domain binding optimization (DBO) on said druggable ligand of (a) by a method comprising
i. making one or more modifications to one or more features at a first receptor binding surface of the druggable ligand to disrupt binding of the druggable ligand to a first target receptor domain, and
ii. making one or more modifications to one or more features at a second receptor binding surface of the druggable ligand to enhance binding of the druggable ligand to a second target receptor domain, and
c) assaying the optimized druggable ligands of (b) for dominant negative activity wherein the dominant negative activity is the inhibition a biological activity.

2. The method of claim 1 wherein the biological activity is selected from the group consisting of a receptor-mediated pathology, receptor-mediated cell signaling, cell growth, cell proliferation and tumor growth.

3. The method of claim 2 further comprising the step of identifying druggable ligands capable of inhibiting a biological activity as therapeutic dominant negative ligands.

4. The method of claim 1 further comprising making modifications to one or more features of the druggable ligands to alter one or more properties of the druggable ligands, said properties selected from the group consisting of optimal pH or pH-activity, digestibility, antigenicity, the amphipathic properties, ligand-receptor interactions, thermal or kinetic stability, solubility, folding, posttranslational modification, hydrophobicity and hydrophilicity.

5. The method of claim 1 wherein the disruption or enhancement of binding of the druggable ligand to a said first or a said second target receptor domain is determined by measuring the binding affinity of the druggable ligand to one or more molecules selected from the group consisting of native target receptors containing the target receptor domain, isolated target receptor domains and representative target receptor moieties.

6. The method of claim 1 wherein said first and said second target receptor domains are located in the same receptor.

7. The method of claim 1 wherein the target receptor is selected from the group consisting of HER receptors, insulin receptors, IGF receptors, interferon receptors, hGH receptors, VEGF receptors, NGF receptors, TNF receptors and G-protein coupled receptors.

8. The method of claim 1 wherein the target receptor is membrane bound.

9. The method of claim 1 wherein the modifications made result in or from the production of a library of modified polypeptides.

10. The method of claim 9 wherein the library of modified polypeptides comprises a phage library.

11. The method of claim 1 wherein binding is determined using phage ELISA.

12. The method of claim 2 wherein the inhibited biological activity is receptor-mediated cell signaling.

13. The method of claim 12 wherein the inhibition of receptor-mediated cell signaling results in ablation of downstream signaling by a receptor as measured by altered phosphorylation states of one or more proteins.

14. The method of claim 12 wherein inhibition of receptor-mediated cell signaling is measured using autophosphorylation assays or gene expression assays.

15. The method of claim 2 wherein the inhibition of biological activity is panoramic over two or more receptors.

16. The method of claim 15 wherein the level or degree panoramic inhibition of biological activity is substantially the same against said two or more receptors.

17. The method of claim 1 wherein the one or more modifications are selected from the group consisting of randomization of one or more features, duplication of one or more features, alteration of length, alteration of electronic charge, and any combination thereof.

18. The method of claim 1 wherein the one or more features are selected from the group consisting of surface manifestations, local conformational shape, fold, loops, half-loops, domains, half-domains, sites and termini.

19. The method of claim 1 further comprising the step of rational redesign wherein steps (a) and (b) are performed iteratively, either alone or in combination.

20. The method of claim 17 wherein the alteration of length is a truncation.

21-22. (canceled)

23. The method of claim 2 wherein the inhibited biological activity is the cause of a receptor-mediated pathology.

24. The method of claim 23 wherein the receptor-mediated pathology is selected from the group consisting of cancer, inflammation, cardiovascular disease, hyperlipidemia, glucose dysregulation, epilepsy, allergies, chronic pain, Alzheimers disease, metabolic syndrome, cortisol resistance, Crohn disease and Huntington disease.

25. The method of claim 2 wherein the one or more cell lines comprises a cancer cell line.

26. The method of claim 25 wherein the type of cancer of said cancer cell line is selected from the group consisting of lung, breast, liver, heart, bone, blood, colon, brain, skin, kidney, pancreatic, ovarian, uterine and prostate.

27. A method of identifying anticancer agents comprising;
assaying therapeutic DNL or DNL variants designed by the method of claim 1 in a tumor xenograft system wherein a measured reduction in tumor growth rate, tumor size or tumor metastasis represents a positive hit as a candidate cancer therapeutic.


SEQUENCE LISTING (45 sequences)

SEQ ID NO: 1 - protein, 84 aa, Artificial Sequence [sequence omitted]

SEQ ID NO: 2 - protein, 53 aa, Artificial Sequence
  Ligand Variant
SHLVKCPLSHDGYCLHDGVCMYIEALDKYACNCVVGYIGERCQYRDLKWWELR

SEQ ID NO: 3 - protein, 53 aa, Artificial Sequence
  Ligand Variant
NWVSECPLSHDGYCLHDGVCMYIEALDKYACNCVVGYIGERCQYRDLKWWELR

SEQ ID NO: 4 - protein, 55 aa, Artificial Sequence
  Ligand Variant
VVSHFNDCPLSHDGYCLHDGVCMYIEALDKYACNCVVGYIGERCQYRDLKWWELR

SEQ ID NO: 5 - protein, 53 aa, Artificial Sequence
  Ligand Variant
NWVSECPLSHDGYCLHDGVCMYIEALDKYACNCVVGYIGEDCQYRDGKWWELR

SEQ ID NO: 6 - protein, 53 aa, Artificial Sequence
  Ligand Variant
NWVSECPLSHDGYCLHDGVCMYIEALDKYACNCVVGYIGEDCQYRDLKWWELR

SEQ ID NO: 7 - protein, 53 aa, Artificial Sequence
  Ligand Variant
NWVSECPLSHDGYCLHDGVCMYIEALDKYACNCVVGYIGERCQYRDGKWWELR

SEQ ID NO: 8 - protein, 53 aa, Artificial Sequence
  Ligand Variant
NWVSECPLSHDGYCLHDGVCRAVARLDKYACNCVVGYIGEDCQYRDGKWWELR

SEQ ID NO: 9 - protein, 53 aa, Artificial Sequence
  Ligand Variant
NWVSECPLSHDGYCLHDGVCKNYNELDKYACNCVVGYIGEDCQYRDGKWWELR

SEQ ID NO: 10 - protein, 53 aa, Artificial Sequence
  Ligand Variant
NWVSECPLSHDGYCLHDGVCYAKGGLDKYACNCVVGYIGEDCQYRDGKWWELR

SEQ ID NO: 11 - protein, 53 aa, Artificial Sequence
  Ligand Variant
NWVSECPLSHDGYCLHDGVCGGGKALDKYACNCVVGYIGEDCQYRDGKWWELR

SEQ ID NO: 12 - protein, 53 aa, Artificial Sequence
  Ligand Variant
NWVSECPLSHDGYCLHDGVCGGSKGLDKYACNCVVGYIGEDCQYRDGKWWELR

SEQ ID NO: 13 - protein, 53 aa, Artificial Sequence
  Ligand Variant
NWVSECPLSHDGYCLHDGVCRERTGLDKYACNCVVGYIGEDCQYRDGKWWELR

SEQ ID NO: 14 - protein, 53 aa, Artificial Sequence
  Ligand Variant
NWVSECPLSHDGYCLHDGVCPRTAPLDKYACNCVVGYIGEDCQYRDGKWWELR

SEQ ID NO: 15 - protein, 53 aa, Artificial Sequence
  Ligand Variant
NWVSECPLSHDGYCLHDGVCTTQTPLDKYACNCVVGYIGEDCQYRDGKWWELR

SEQ ID NO: 16 - protein, 53 aa, Artificial Sequence
  Ligand Variant
NWVSECPLSHDGYCLHDGVCTNKERLDKYACNCVVGYIGEDCQYRDGKWWELR

SEQ ID NO: 17 - protein, 53 aa, Artificial Sequence
  Ligand Variant
NWVSECPLSHDGYCLHDGVCSGRPTLDKYACNCVVGYIGEDCQYRDGKWWELR

SEQ ID NO: 18 - protein, 53 aa, Artificial Sequence
  Ligand Variant
NWVSECPLSHDGYCLHDGVCMGMGRLDKYACNCVVGYIGEDCQYRDGKWWELR

SEQ ID NO: 19 - protein, 53 aa, Artificial Sequence
  Ligand Variant
NWVSECPLSHDGYCLHDGVCMGSSGLDKYACNCVVGYIGEDCQYRDGKWWELR

SEQ ID NO: 20 - protein, 53 aa, Artificial Sequence
  Ligand Variant
NWVSECPLSHDGYCLHDGVCTTNKALDKYACNCVVGYIGEDCQYRDGKWWELR

SEQ ID NO: 21 - protein, 53 aa, Artificial Sequence
  Ligand Variant
NWVSECPLSHDGYCLHDGVCKPEKQLDKYACNCVVGYIGEDCQYRDGKWWELR

SEQ ID NO: 22 - protein, 53 aa, Artificial Sequence
  Ligand Variant
NWVSECPLSHDGYCLHDGVCDNPMRLDKYACNCVVGYIGEDCQYRDGKWWELR

SEQ ID NO: 23 - protein, 53 aa, Artificial Sequence
  Ligand Variant
NWVSECPLSHDGYCLHDGVCGPQAPLDKYACNCVVGYIGEDCQYRDGKWWELR

SEQ ID NO: 24 - protein, 53 aa, Artificial Sequence
  Ligand Variant
NWVSECPLSHDGYCLHDGVCMYIEAHPKSYCNCVVGYIGEDCQYRDGKWWELR

SEQ ID NO: 25 - protein, 53 aa, Artificial Sequence
  Ligand Variant
NWVSECPLSHDGYCLHDGVCMYIEATPSYLCNCVVGYIGEDCQYRDGKWWELR

SEQ ID NO: 26 - protein, 53 aa, Artificial Sequence
  Ligand Variant
NWVSECPLSHDGYCLHDGVCMYIEANREKTCNCVVGYIGEDCQYRDGKWWELR

SEQ ID NO: 27 - protein, 53 aa, Artificial Sequence
  Ligand Variant
NWVSECPLSHDGYCLHDGVCMYIEASKRQPCNCVVGYIGEDCQYRDGKWWELR

SEQ ID NO: 28 - protein, 53 aa, Artificial Sequence
  Ligand Variant
NWVSECPLSHDGYCLHDGVCMYIEAQIKLLCNCVVGYIGEDCQYRDGKWWELR

SEQ ID NO: 29 - protein, 53 aa, Artificial Sequence
  Ligand Variant
NWVSECPLSHDGYCLHDGVCMYIEAGTKHRCNCVVGYIGEDCQYRDGKWWELR

SEQ ID NO: 30 - protein, 53 aa, Artificial Sequence
  Ligand Variant
NWVSECPLSHDGYCLHDGVCMYIEAISLRSCNCVVGYIGEDCQYRDGKWWELR

SEQ ID NO: 31 - protein, 53 aa, Artificial Sequence
  Ligand Variant
NWVSECPLSHDGYCLHDGVCMYIEAGTARGCNCVVGYIGEDCQYRDGKWWELR

SEQ ID NO: 32 - protein, 53 aa, Artificial Sequence
  Ligand Variant
NWVSECPLSHDGYCLHDGVCMYIEAENKRRCNCVVGYIGEDCQYRDGKWWELR

SEQ ID NO: 33 - protein, 53 aa, Artificial Sequence
  Ligand Variant
NWVSECPLSHDGYCLHDGVCMYIEAYGNTTCNCVVGYIGEDCQYRDGKWWELR

SEQ ID NO: 34 - protein, 53 aa, Artificial Sequence
  Ligand Variant
NWVSECPLSHDGYCLHDGVCMYIEADRSLTCNCVVGYIGEDCQYRDGKWWELR

SEQ ID NO: 35 - protein, 53 aa, Artificial Sequence
  Ligand Variant
NWVSECPLSHDGYCLHDGVCMYIEASHGQECNCVVGYIGEDCQYRDGKWWELR

SEQ ID NO: 36 - protein, 53 aa, Artificial Sequence
  Ligand Variant
NWVSECPLSHDGYCLHDGVCMYIEAQIAGACNCVVGYIGEDCQYRDGKWWELR

SEQ ID NO: 37 - protein, 53 aa, Artificial Sequence
  Ligand Variant
NWVSECPLSHDGYCLHDGVCMYIEAPNPSPCNCVVGYIGEDCQYRDGKWWELR

SEQ ID NO: 38 - protein, 53 aa, Artificial Sequence
  Ligand Variant
NWVSECPLSHDGYCLHDGVCMYIEAGKSMKCNCVVGYIGEDCQYRDGKWWELR

SEQ ID NO: 39 - protein, 53 aa, Artificial Sequence
  Ligand Variant
NWVSECPLSHDGYCLHDGVCMYIEAQPHLSCNCVVGYIGEDCQYRDGKWWELR

SEQ ID NO: 40 - protein, 53 aa, Artificial Sequence
  Ligand Variant
NWVSECPLSHDGYCLHDGVCMYIEAPHASLCNCVVGYIGEDCQYRDGKWWELR

SEQ ID NO: 41 - protein, 53 aa, Artificial Sequence
  Ligand Variant
NWVSECPLSHDGYCLHDGVCMYIEAQMQSRCNCVVGYIGEDCQYRDGKWWELR

SEQ ID NO: 42 - protein, 53 aa, Artificial Sequence
  Ligand Variant
NWVSECPLSHDGYCLHDGVCMYIEAYRVKTCNCVVGYIGERCQYRDLKWWELR

SEQ ID NO: 43 - protein, 53 aa, Artificial Sequence
  Ligand Variant
NWVSECPLSHDGYCLHDGVCYRVKTLDKYACNCVVGYIGERCQYRDLKWWELR

SEQ ID NO: 44 - protein, 53 aa, Artificial Sequence
  Ligand Variant
NWVSECPLSHDGYCLHDGVCMYIEATKYRGCNCVVGYIGERCQYRDLKWWELR

SEQ ID NO: 45 - protein, 101 aa, Artificial Sequence
  IGF Clone
MKKTAIAIAVALAGFATVAQAHHHHHHIEGRGPETLCGAELVDALQFVCGDKGFYFNKPTGYGSSSRRAPQTGIVDECCFRSCDLRRLEMYCAPLKPAKSA